SEQUENCE LISTING (21 sequences)

SEQ ID NO: 1 - protein, 267 aa, Neonothopanus nambi
MRINISLSSLFERLSKLSSRSIAITCGVVLASAIAFPIIRRDYQTFLEVGPSYAPQNFRGYIIVCVLSLFRQEQKGLAIYDRLPEKRRWLADLPFREGTRPSITSHIIQRQRTQLVDQEFATRELIDKVIPRVQARHTDKTFLSTSKFEFHAKAIFLLPSIPINDPLNIPSHDTVRRTKREIAHMHDYHDCTLHLALAAQDGKEVLKKGWGQRHPLAGPGVPGPPTEWTFLYAPRNEEEARVVEMIVEASIGYMTNDPAGKIVENAK

SEQ ID NO: 2 - protein, 267 aa, Neonothopanus nambi
MRINISLSSLFERLSKLSSRSIAITCGVVLASAIAFPIIRRDYQTFLEVGPSYAPQNFRGYIIVCVLSLFRQEQKGLAIYDRLPEKRRWLADLPFREGPRPSITSHIIQRQRTQLVDQEFATRELIDKVIPRVQARHTDKTFLSTSKFEFHAKAIFLLPSIPINDPLNIPSHDTVRRTKREIAHMHDYHDCSLHLALAPQDGKEVLKKGWGQRHPLAGPGVPGPPTEWTFLYAPRNEEEARVVEMIVEASIGYMTNDPAGKIVENAK

SEQ ID NO: 3 - protein, 267 aa, Neonothopanus nambi
MRSTISLSSLLERLSKLSSRSIAITCGVVLASAIAFPIIRRDYQTFLEVGPSYAPQNFRGYITVCVLSLFRQEQKGLAIYDRLPEKRRWLADLPFREGPRPSITSHIIQRQRTQLVDQEFATRELIDKVIPRVQARHTDKTFLSTSKFEFHAKAIFLLPSIPINDPLNIPSHDTVRRTKREIAHMHDYHDCSLHLALAPQDGKEVLKKGWGQRHPLAGPGVPGPPTEWTFLYAPRNEEEARVVEMIVEASIGYMTNDPAGKIVENAK

SEQ ID NO: 4 - protein, 269 aa, Neonothopanus nambi
MRSTISLSSLLERLSKLSSRSIAITCGVVLASAIAFPIIRRDYQTFLEVGPSYAPQNFRGYITVCVLSLFRQEQKGLAIYDRLPEKRRWLADLPFREGPRPSITSHIIQRQRTQLVDQEFATRELIDKVIPRVQARHTDKTFLSTSKFEFHAKAIFLLPSIPINDPLNIPSHDTVRRTKREIAHMHDYHDCSLHLALAPQDGKEVLKKGWGQRHPLAGPGVPGPPTEWTFLYAPRNEEEARVVEMIVEASIGYMTNDPAGKIVENAKGS

SEQ ID NO: 5 - protein, 422 aa, Neonothopanus nambi
MASFENSLSVLIVGAGLGGLAAAIALRRQGHVVKIYDSSSFKAELGAGLAVPPNTLRSLQQLGCNTENLNGVDNLCFTAMGYDGSVGMMNNMTDYREAYGTSWIMVHRVDLHNELMRVALDPGGLGPPATLHLNHRVTFCDVDACTVTFTNGTTQSADLIVGADGIRSTIRRFVLEEDVTVPASGIVGFRWLVQADALDPYPELDWIVKKPPLGARLISTPQNPQSGVGLADRRTIIIYACRGGTMVNVLAVHDDERDQNTADWSVPASKDDLFRVFHDYHPRFRRLLELAQDINLWQMRVVPVLKKWVNKRVCLLGDAAHASLPTLGQGFGMGLEDAVALGTLLPKGTTASQIETRLAVYEQLRKDRAEFVAAESYEEQYVPEMRGLYLRSKELRDRVMGYDIKVESEKVLETLLRSSNSA

SEQ ID NO: 6 - protein, 422 aa, Neonothopanus nambi
MASFENSLSVLIVGAGLGGLAAAIALRRQGHVVKIYESSSFKAELGAGLAVPPNTLRSLQQLGCNTENLNGVDNLCFTAMGYDGSVGMMNNMTDYREAYGTSWIMVHRVDLHNELMRIALDPGGLGPPATLHLNHRVTFCDVDACTVTFTNGTTQSADLIVGADGIRSTIRRFVLEEDVTVPASGIVGFRWLVQADALDPYPELDWIVKKPPLGARLISTPQNPQSGVGLADRRTIIIYACRGGTMVNVLAVHDDERDQNTADWSVPASKDDLFRVFHDYHPRFRRLLELAQDINLWQMRVVPVLKKWVNKRVCLLGDAAHAMLPTLGQGFGMGLEDAVALGTLLPKGTTASQIETRLAVYEQLRKDRAEFVAAESYEEQYVPEKRGLYLRSKELRDRVMGYDIKVESEKVLETLLRSSNSA

SEQ ID NO: 7 - protein, 424 aa, Neonothopanus nambi
MASFENSLSVLIVGAGLGGLAAAIALRRQGHVVKIYESSSFKAELGAGLAVPPNTLRSLQQLGCNTENLNGVDNLCFTAMGYDGSVGMMNNMTDYREAYGTSWIMVHRVDLHNELMRIALDPGGLGPPATLHLNHRVTFCDVDACTVTFTNGTTQSADLIVGADGIRSTIRRFVLEEDVTVPASGIVGFRWLVQADALDPYPELDWIVKKPPLGARLISTPQNPQSGVGLADRRTIIIYACRGGTMVNVLAVHDDERDQNTADWSVPASKDDLFRVFHDYHPRFRRLLELAQDINLWQMRVVPVLKKWVNKRVCLLGDAAHAMLPTLGQGFGMGLEDAVALGTLLPKGTTASQIETRLAVYEQLRKDRAEFVAAESYEEQYVPEKRGLYLRSKELRDRVMGYDIKVESEKVLETLLRSSNSAGS

SEQ ID NO: 8 - DNA, 801 bp, Neonothopanus nambi
  source 1..801: mol_type = genomic DNA
atgcgcattaacattagcctctcgtctctcttcgaacgtctctccaaacttagcagtcgcagcatagcgattacatgtggagttgttctcgcctccgcaatcgcctttcccatcatccgcagagactaccagactttcctagaagtgggaccctcgtacgctccgcagaactttagaggatacatcatcgtctgtgtcctctcgctattccgccaagagcagaaagggctcgccatctatgatcgtcttcccgagaaacgcaggtggttggccgaccttccctttcgtgaaggaaccagacccagcattaccagccatatcattcagcgacagcgcactcaactggtcgatcaggagtttgccaccagggagctcatagacaaggtcatccctcgcgtgcaagcacgacacaccgacaaaacgttcctcagcacatcaaagttcgagtttcatgcgaaggccatatttctcttgccttctatcccaatcaacgaccctctgaatatccctagccacgacactgtccgccgaacgaagcgcgagattgcacatatgcatgattatcatgattgcacacttcatcttgctctcgctgcgcaggatggaaaggaggtgctgaagaaaggttggggacaacgacatcctttggctggtcctggagttcctggtccaccaacggaatggacttttctttatgcgcctcgcaacgaagaagaggctcgagtagtggagatgatcgttgaggcttccatagggtatatgacgaacgatcctgcaggaaagattgtagaaaacgccaag

SEQ ID NO: 9 - DNA, 801 bp, Neonothopanus nambi
  source 1..801: mol_type = genomic DNA
atgcgcattaacattagcctctcgtctctcttcgaacgtctctccaaacttagcagtcgcagcatagcgattacatgtggagttgttctcgcctccgcaatcgcctttcccatcatccgcagagactaccagactttcctagaagtgggaccctcgtacgctccgcagaactttagaggatacatcatcgtctgtgtcctctcgctattccgccaagagcagaaagggctcgccatctatgatcgtcttcccgagaaacgcaggtggttggccgaccttccctttcgtgaaggaccaagacccagcattaccagccatatcattcagcgacagcgcactcaactggtcgatcaggagtttgccaccagggagctcatagacaaggtcatccctcgcgtgcaagcacgacacaccgacaaaacgttcctcagcacatcaaagttcgagtttcatgcgaaggccatatttctcttgccttctatcccaatcaacgaccctctgaatatccctagccacgacactgtccgccgaacgaagcgcgagattgcacatatgcatgattatcatgattgcagccttcatcttgctctcgctccacaggatggaaaggaggtgctgaagaaaggttggggacaacgacatcctttggctggtcctggagttcctggtccaccaacggaatggacttttctttatgcgcctcgcaacgaagaagaggctcgagtagtggagatgatcgttgaggcttccatagggtatatgacgaacgatcctgcaggaaagattgtagaaaacgccaag

SEQ ID NO: 10 - DNA, 801 bp, Neonothopanus nambi
  source 1..801: mol_type = genomic DNA
atgcgcagtaccattagcctctcgtctctcctcgaacgtctctccaaacttagcagtcgcagcatagcgattacatgtggagttgttctcgcctccgcaatcgcctttcccatcatccgcagagactaccagactttcctagaagtgggaccctcgtacgctccgcagaactttagaggatacatcaccgtctgtgtcctctcgctattccgccaagagcagaaagggctcgccatctatgatcgtcttcccgagaaacgcaggtggttggccgaccttccctttcgtgaaggaccaagacccagcattaccagccatatcattcagcgacagcgcactcaactggtcgatcaggagtttgccaccagggagctcatagacaaggtcatccctcgcgtgcaagcacgacacaccgacaaaacgttcctcagcacatcaaagttcgagtttcatgcgaaggccatatttctcttgccttctatcccaatcaacgaccctctgaatatccctagccacgacactgtccgccgaacgaagcgcgagattgcacatatgcatgattatcatgattgcagccttcatcttgctctcgctccacaggatggaaaggaggtgctgaagaaaggttggggacaacgacatcctttggctggtcctggagttcctggtccaccaacggaatggacttttctttatgcgcctcgcaacgaagaagaggctcgagtagtggagatgatcgttgaggcttccatagggtatatgacgaacgatcctgcaggaaagattgtagaaaacgccaag

SEQ ID NO: 11 - DNA, 807 bp, Neonothopanus nambi
  source 1..807: mol_type = genomic DNA
atgaggtctactatctctttgtctagtctccttgagagactgagcaaattgtcatcccgatccatcgcaattacctgcggcgtagtcttggcaagtgcaatagcattcccaattatccgaagagactatcaaacgttccttgaagtcggtccgagctatgccccacagaacttccgaggctatatcactgtttgtgtcttgtcactttttaggcaagagcagaaagggttggcaatctatgacaggcttccagaaaagaggcgttggcttgccgacttgccgtttcgtgaagggccaaggccatccataacctcccacattatacagcgtcagcgtactcagctggtagatcaagagtttgcaaccagagaacttatcgataaggtgatcccacgtgtgcaagcaagacacacagacaaaacttttctcagtacgtcaaaatttgagtttcatgcaaaggccatcttccttctcccctctatccctattaatgatcctcttaacattccctcacacgatacggtaagaagaaccaagcgtgagatcgctcacatgcatgattaccatgattgctctctgcacttggctcttgctcctcaggatggtaaggaagttttgaagaaggggtggggccagcgtcacccactggccggaccaggagtcccaggccctcctactgagtggaccttcctttacgcaccaaggaacgaagaggaggccagagttgtcgagatgatagtcgaagctagtattggctacatgactaacgatcctgctggtaagattgtcgaaaatgccaaaggtagt

SEQ ID NO: 12 - DNA, 1266 bp, Neonothopanus nambi
  source 1..1266: mol_type = genomic DNA
atggcatcgtttgagaattctctaagcgttttgattgtcggggccggacttggtgggcttgctgctgccatcgcgctgcgtcgccaagggcatgtcgtgaaaatatacgactcctctagcttcaaagccgaacttggtgcgggactcgctgtgccgcctaacaccttgcgcagtctacagcaacttggttgcaataccgagaacctcaatggtgtggataatctttgcttcactgcgatggggtatgacgggagtgtagggatgatgaacaacatgactgactatcgagaggcatacggtacttcttggatcatggtccaccgcgttgacttgcataacgagctgatgcgcgtagcacttgatccaggtgggctcggacctcctgcgacactccatcttaatcatcgtgtcacattctgcgatgtcgacgcttgcaccgtgacattcaccaacgggaccactcaatcagctgatctcatcgttggtgcagacggtatacgctctaccattcggcggtttgtcttagaagaagacgtgactgtgcctgcgtcaggaatcgtcgggtttcgatggcttgtacaagctgacgcgctggacccatatcctgaactcgactggattgttaaaaagcctcctctaggcgcgcgactgatctccactcctcagaatccacagtctggtgttggcttggctgacaggcgcactatcatcatctacgcatgtcgtggcggcaccatggtcaatgtccttgcagtgcatgatgacgaacgtgaccagaacaccgcagattggagtgtaccggcttccaaagacgatctatttcgtgttttccacgattaccatccacgctttcggcggcttttagagcttgcgcaggatattaatctctggcaaatgcgtgttgtacctgttttgaaaaaatgggttaacaagcgggtttgcttgttaggagatgctgcgcacgcttctttaccgacgttgggtcaaggttttggtatgggtctggaagatgccgtagcacttggtacactccttccaaagggtaccactgcatctcagatcgagactcgacttgcggtgtacgaacagctacgtaaggatcgtgcggaatttgttgcggctgaatcatatgaagagcaatatgttcctgaaatgcggggactttatctgaggtcaaaggaactgcgtgatagagtcatgggttatgatatcaaagtggagagcgagaaggttctcgagacgctcctaagaagttctaattctgcc

SEQ ID NO: 13 - protein, 699 aa, Neonothopanus nambi
MRSTISLSSLLERLSKLSSRSIAITCGVVLASAIAFPIIRRDYQTFLEVGPSYAPQNIRGYITVCVLSLFRQEQKGLAIYDRLPEKRRWLADLPFRVGPRPSITSHIIQRQRTQLVDQEFATRELIDKVIPRVQARHTDKTFLSTSKFEFHAKAIFLLPSIPINDPLNIPSHDTVRRTKREIAHMHDYHDCSLHLALAPQDAKEVLKKGWGQRHPLAGPGVPGPPTEWTFLYAPRNEEEARVVEMIVEASIGYMTNDPAGKIVENAKGGSGGGGSLGMASFENSLSVLIVGAGLGGLAAAIALRRQGHVVKIYESSSFKAELGAGLAVPPNTLRSLQQLGCNTENLNGVDNLCFTAMGYDGSVGMMNNMTDYREAYGTSWIMVHRVDLHNELMRVALDPGGLGPPATLHLNHRVTFCDVDACTVTFTNGTTQSADLIVGADGIRSTIRRFVLEEDVTIPASGIAGFRWLVQADALDPYPELDWIVKKPPLGARLISTPQNPQSGVGLADRRTIIIYACRGGTMVNVLAVHDDERDQNTADWSVPASKDDLFRVFHDYHPRFRRLLELAQDINLWQMRVVPVLKKWVNKRVCLLGDAAHAMLPTLGQGFGMGLEDAVALGTLLPKSTTASQIETRLAVYEQLRKDRAEFVAAESYEEQYVPEKRGLYLRSKELRDRVMGYDIKVESEKVLETLLRSSNSA

SEQ ID NO: 14 - protein, 267 aa, Neonothopanus nambi
MRSTISLSSLLERLSKLSSRSIAITCGVVLASAIAFPIIRRDYQTFLEVGPSYAPQNIRGYITVCVLSLFRQEQKGLAIYDRLPEKRRWLADLPFRVGPRPSITSHIIQRQRTQLVDQEFATRELIDKVIPRVQARHTDKTFLSTSKFEFHAKAIFLLPSIPINDPLNIPSHDTVRRTKREIAHMHDYHDCSLHLALAPQDAKEVLKKGWGQRHPLAGPGVPGPPTEWTFLYAPRNEEEARVVEMIVEASIGYMTNDPAGKIVENAK

SEQ ID NO: 15 - protein, 422 aa, Neonothopanus nambi
MASFENSLSVLIVGAGLGGLAAAIALRRQGHVVKIYESSSFKAELGAGLAVPPNTLRSLQQLGCNTENLNGVDNLCFTAMGYDGSVGMMNNMTDYREAYGTSWIMVHRVDLHNELMRVALDPGGLGPPATLHLNHRVTFCDVDACTVTFTNGTTQSADLIVGADGIRSTIRRFVLEEDVTIPASGIAGFRWLVQADALDPYPELDWIVKKPPLGARLISTPQNPQSGVGLADRRTIIIYACRGGTMVNVLAVHDDERDQNTADWSVPASKDDLFRVFHDYHPRFRRLLELAQDINLWQMRVVPVLKKWVNKRVCLLGDAAHAMLPTLGQGFGMGLEDAVALGTLLPKSTTASQIETRLAVYEQLRKDRAEFVAAESYEEQYVPEKRGLYLRSKELRDRVMGYDIKVESEKVLETLLRSSNSA

SEQ ID NO: 16 - protein, 266 aa, Neonothopanus nambi
MLIYFRSIVEFFSGASGRSAAITCVLVLTSAIALPFIRKDYQTFLEVGPSYAPQNFKGYIIVCVLALFRQEQKGLAIYDHLPEKRRWLEDLPYRDGSRPSITSHIIQRQRTQLVEPDFATKNLIGKVIPRVQARHTDKTFLSTSKFEFHAEAIFLSLSIPINDPQNIPSHDTVRRTKREIAHMHDYHDCTLHLALAAQDGKEVLRKGWGQRHPLAGPGMPGPPTEWTFLYAPRNEKEVRIVEMIVEASVGYMTNDPAGKIVESKEE

SEQ ID NO: 17 - protein, 251 aa, Neonothopanus nambi
MVQLTRTSGFIAAAAIVAAIAFPFIRRDYQTFLRGGPSYAPQNIRGYIIVLVLSLFRGEEKGLAIYEPLPEKRTWLPELPRRAGDRPKTTSHIIQRQLDQYPDPDFVLKALKATVIPRVQARHTDKTHLALSKFEFHAEAIFVRPEIAIDDPKHIPSHDTVRRTKREIAHMHDYHDCTLHLALAAQDAKQVLQKGWGQRHPLAGPGMPGPPTEWTFLYAPRTEEEVKVVETIVEASIAYMTNAEKPVELVQ

SEQ ID NO: 18 - protein, 426 aa, Neonothopanus nambi
MQQIDEAHPLTVIVVGAGLCGLSAAIALRRQGHHVHILESSSFKSELGAGLAVPPNTVRSLRGLGCNIDNLKPVDNLCFTAMAHDGSPGMMNNMTDYHKAYGEPWVMAHRVDLHNELMRVALDPEGTGPPAQLRLGVQVVSCDVEACTISLVDGSICSADLIVGADGIRSTIRSYVLGKEIDIPPTGIAGYRWLTPAEALEPYPELDWIIKNPPLGARLITAPVRRNDKTDDGEKTDKRTIIIYACRNGTMINVLGVHDDPRNQNEVGWNVPVTQENLLEFFEDYHPRFKRLLQLADSIHLWQMRVVPRLDTWINKRVCLLGDSAHASLPTLGQGFGMGLEDAVALAALLPMGTKVSDVESRLIAYESLRKERAEYVATESLEQQNIPGKRGLYLRSPMMRDKIMGYDIKAEAEKVLMELITSTAQ

SEQ ID NO: 19 - protein, 708 aa, Neonothopanus nambi
MLIYFRSIVEFFSGASGRSAAITCVLVLTSAIALPFIRKDYQTFLEVGPSYAPQNFKGYIIVCVLALFRQEQKGLAIYDHLPEKRRWLEDLPYRDGSRPSITSHIIQRQRTQLVEPDFATKNLIGKVIPRVQARHTDKTFLSTSKFEFHAEAIFLSLSIPINDPQNIPSHDTVRRTKREIAHMHDYHDCTLHLALAAQDGKEVLRKGWGQRHPLAGPGMPGPPTEWTFLYAPRNEKEVRIVEMIVEASVGYMTNDPAGKIVESKEEGGGGGSGGGGSCGGGSMQQIDEAHPLTVIVVGAGLCGLSAAIALRRQGHHVHILESSSFKSELGAGLAVPPNTVRSLRGLGCNIDNLKPVDNLCFTAMAHDGSPGMMNNMTDYHKAYGEPWVMAHRVDLHNELMRVALDPEGTGPPAQLRLGVQVVSCDVEACTISLVDGSICSADLIVGADGIRSTIRSYVLGKEIDIPPTGIAGYRWLTPAEALEPYPELDWIIKNPPLGARLITAPVRRNDKTDDGEKTDKRTIIIYACRNGTMINVLGVHDDPRNQNEVGWNVPVTQENLLEFFEDYHPRFKRLLQLADSIHLWQMRVVPRLDTWINKRVCLLGDSAHASLPTLGQGFGMGLEDAVALAALLPMGTKVSDVESRLIAYESLRKERAEYVATESLEQQNIPGKRGLYLRSPMMRDKIMGYDIKAEAEKVLMELITSTAQ

SEQ ID NO: 20 - protein, 708 aa, Neonothopanus nambi
MLIYFRSIVEFFSGASGRSAAITCVLVLTSAIALPFIRKDYQTFLEVGPSYAPQNFKGYIIVCVLALFRQEQKGLAIYDHLPEKRRWLEDLPYRDGSRPSITSHIIQRQRTQLVEPDFATKNLIGKVIPRVQARHTDKTFLSTSKFEFHAEAIFLSLSIPINDPQNIPSHDTVRRTKREIAHMHDYHDCTLHLALAAQDGKEVLRKGWGQRHPLAGPGMPGPPTEWTFLYAPRNEKEVRIVEMIVEASVGYMTNDPAGKIVESKEEGGGGGSGGGGSCGGGSMQQIDEAHPLTVIVVGAGLCGLSAAIALRRQGHHVHILESSSFKSELGAGLAVPPNTVRSLRGLGCNIDNLKPVDNLCFTAMAHDGSPGMMNNMTDYHKAYGEPWVMAHRVDLHNELMRVALDPEGTGPPAQLRLGVQVVSCDVEACTISLVDGTICSADLIVGADGIRSTIRSYVLGKEIDIPPTGIAGYRWLTPAEALEPYPELDWIIKNPPLGARLITAPVRRNDKTDDGEKTDKRTIIIYACRNGTMINVLGVHDDPRNQNEVGWNVPVTQENLLEFFEDYHPRFKRLLQLADSIHLWQMRVVPRLDTWINKRVCLLGDSAHASLPTLGQGFGMGLEDAVALAALLPMGTKVSDVESRLIAYESLRKERAEYVATESLEQQNIPGKRGLYLRSPMMRDKIMGYDIKAEAEKVLMELITSTAQ

SEQ ID NO: 21 - protein, 708 aa, Neonothopanus nambi
MLIYFRSIVEFFSGASGRSAAITCVLVLTSAIALPFIRKDYQTFLEVGPSYAPQNFKGYIIVCVLALFRQEQKGLAIYDHLPEKRRWLEDLPYRDGSRPSITSHIIQRQRTQLVEPDFATKNLIGKVIPRVQARHTDKTFLSTSKFEFHAEAIFLSLSIPINDPQNIPSHDTVRRTKREIAHMHDYHDCTLHLALAAQDGKEVLRKGWGQRHPLAGPGMPGPPTEWTFLYAPRNEKEVRIVEMIVEASVGYMTNDPAGKIVESKEEGGGGGSGGGGSCGGGSMQQIDEAHPLTVIVVGAGLCGLSAAIALRRQGHHVHILESSSFKSELGAGLAVPPNTVRSLRGLGCNIDNLKPVDNLCFTAMAHDGSPGMMNNMTDYHKAYGEPWVMAHRVDLHNELMRVALDPEGTGPPAQLRLGVQVVSCDVEACTISLVDGSICSADLIVGADGIRSTIRSYVLGKEIDIPPTGIAGYRWLTPAEALEPYPELDWIIKNPPLGARLITAPVRRNDKTDDGEKTDKRTIIIYACRNGTMINVLGVHDDPRNQNEVGWNVPVTQENLLEFFEDYHPRFKRLLQLADSIHLWQMRVVPRLDTWINKRVCLLGDSAHASLPTLGQGFGMGLEDAVALAALLPMSTKVSDVESRLIAYESLRKERAEYVATESLEQQNIPGKRGLYLRSPMMRDKIMGYDIKAEAEKVLMELITSTAQ

(19) United States
(12) Patent Application Publication
(10) Pub. No.: US 2025/0075191 A1
(43) Pub. Date: Mar. 6, 2025

(54) MODIFIED BIOLUMINESCENT ENZYMES

(71) Applicant: Light Bio, Inc., Ketchum, ID (US)

(72) Inventors: Lance P. Encell, Madison, WI (US); Mary P. Hall, Madison, WI (US); Trish T. Hoang, Madison, WI (US); Keith V. Wood, Ketchum, ID (US); Monika G. Wood, Madison, WI (US); Alexander S. Mishin, Moscow (RU); Karen S. Sarkisyan, Moscow (RU); Ilia V. Yampolsky, Moscow (RU)

(73) Assignee: Light Bio, Inc., Ketchum, ID (US)

(21) Appl. No.: 18/816,641

(22) Filed: Aug. 27, 2024

Related U.S. Application Data
(60) Provisional application No. 63/534,957, filed on Aug. 28, 2023.
(60) Provisional application No. 63/619,077, filed on Jan. 9, 2024.

(51) Int. Cl. C12N 9/10; C12N 9/02

(52) U.S. Cl. CPC C12N 9/1029; C12N 9/0004

(57) ABSTRACT

One or more new variant(s) of luciferases and fungal luciferin biosynthesis enzymes, nucleic acids able to encode these enzymes, and proteins able to catalyze certain stages of the fungal luciferin biosynthesis are provided herein. More specifically one or more bioluminescent enzymes, and in particular one or more hispidin hydroxylases and luciferases, and functional fragments thereof and nucleic acids encoding them are disclosed.

This non-provisional patent application claims all benefits under 35 U.S.C. § 119(e) of pending U.S. provisional patent application Ser. No. 63/534,957 filed 28 Aug. 2023, entitled “Modified Bioluminescent Enzymes”, and pending U.S. provisional patent application Ser. No. 63/619,077 filed 9 Jan. 2024, entitled “Modified Bioluminescent Enzymes”, in the United States Patent and Trademark Office, each of which are incorporated by reference in their entirety herein.

### REFERENCE TO SEQUENCE LISTING SUBMITTED ELECTRONICALLY

The contents of the electronically submitted sequence listing, (Light_Bio_Inc_Sequence_Listing.xml, file created 2024-08-23, 32 KB) filed herewith, is herein incorporated by reference in its entirety.

### FIELD OF THE INVENTION

The present disclosure relates generally to the field of biology and chemistry and more particularly to bioluminescent systems.

### DESCRIPTION OF THE PRIOR ART AND OBJECTIVES OF THE INVENTION

Bioluminescence refers to the ability to emit light through a biochemical process. Several types of bioluminescence are known, which have evolved independently in various organisms. The ability to produce bioluminescence is provided by a specific protein: a luciferase or a photoprotein. Luciferases are enzymes which catalyze the oxidation of low molecular weight compounds, i.e. luciferins, to emit light. The product molecule of the reaction, oxyluciferin, is then released from the enzyme.

Bioluminescence in an organism relies on the availability of the luciferin, usually achieved through endogenous biosynthesis. The structures of the luciferins vary widely among the different types of bioluminescence. In most instances, the full set of proteins (and corresponding genes) needed to synthesize a luciferin is unknown. Only two pathways for luciferin biosynthesis have been discovered: a branch of fatty acid metabolism in marine bacteria (encoded by the lux operon) and a branch of phenylpropanoid metabolism in fungi (the caffeic acid cycle).

The bacterial pathway has been known since the late 1980s; however, it was not widely applied in eukaryotes, likely due to low light output and toxicity of pathway intermediates. In contrast, the discovery of enzymes catalyzing light emission in the fungus Neonothopanus nambi (FIG. 1) has been adapted to other multicellular organisms. In particular, this has enabled autonomous luminescence and reporter tools in plants.

Nonetheless, the utility of this bioluminescence pathway remains limited due to insufficient photon yield in heterologous hosts. This precludes its use in some applications by having greater technical requirements. Thus, in view of the problems and disadvantages associated with prior art, there is a need to further develop the pathway for enhanced bioluminescence in plants, fungi, and mammalian hosts. In view of the foregoing, the present disclosure was conceived and one of its objectives is to provide novel bioluminescent enzymes, in particular novel hispidin hydroxylases and luciferases, and functional fragments thereof and nucleic acids encoding them. The present disclosure also provides novel compositions and methods of use comprising said novel bioluminescent enzymes. The hispidin hydroxylases of the present disclosure catalyze conversion of hispidin into the 3-hydroxyhispidin while the luciferases oxidize 3-hydroxyhispidin resulting in emission of light.

It is another objective of the present disclosure to provide an isolated luciferase or a functional fragment thereof with an amino acid sequence at least 90% identical to the amino acid sequence given in SEQ ID NO: 1 and comprises at least one amino acid substitution selected from T99P, T192S, A199P. One or more embodiments of the isolated luciferase of the present disclosure may have at least 90% sequence identity to positions 40-267 of SEQ ID NO: 1.

It is still another objective of the present disclosure to provide an isolated luciferase further comprises at least one amino acid substitution selected from R2S, I3N, I3S, N4T, F11L, E12D, I63T.

It is yet another objective of the present disclosure to provide an isolated luciferase with an amino acid sequence at least 90% identical to the amino acid sequence given in SEQ ID NO: 1 and comprises the amino acid substitutions T99P, T192S, and A199P.

It is a further objective of the present disclosure to provide an isolated luciferase has an amino acid sequence at least 90% identical to the amino acid sequence given in SEQ ID NO: 1 and comprises the amino acid substitutions I3S, N4T, F11L, I63T, T99P, T192S, and A199P.

It is still a further objective of the present disclosure to provide an isolated luciferase or a functional fragment thereof comprises at least 90%, 91%, 92%, 93%, 94%, 95%, 96%, 97%, 98%, 99% or 100% sequence identity with SEQ ID NO: 1. In some embodiments, the luciferase is present as a fusion to another amino acid sequence (i.e., a fusion protein).

It is yet a further objective of the present disclosure to provide an amino acid sequence of the isolated luciferase or functional fragment thereof comprises at least one amino acid substitution at a position selected from 2, 3, 4, 11, 12, 63, 99, 192, 199 relatives to SEQ ID NO: 1. Some embodiments comprise at least one substitution of the amino acids R2, 13, N4, F11, E12, 163, T99, T192, A199 relatives to SEQ ID NO: 1. Some embodiments further comprise substitutions of amino acids located elsewhere within the sequence.

It is another objective of the present disclosure to provide an amino acid sequence of the isolated luciferase or a functional fragment thereof comprises amino acid substitutions at positions 99, 192, and 199 relatives to SEQ ID NO: 1. Some embodiments comprise substitutions of the amino acids T99, T192, A199 relatives to SEQ ID NO: 1.

It is a further objective of the present disclosure to provide an amino acid sequence of the isolated luciferase, or a functional fragment thereof, that includes at least one substitution of proline at position 99, serine at position 192, or proline at position 199 relative to SEQ ID NO: 1. In some embodiments, one or more additional amino acid substitutions may be present at other positions within the sequence.

It is yet another objective of the present disclosure to provide a luciferase has an amino acid sequence comprising SEQ ID NO: 3.

It is still another objective of the present disclosure to provide one or more nucleic acid molecules encoding luciferases or functional fragments thereof. In some embodiments the nucleic acid molecule encodes a luciferase or a functional fragment thereof having an amino acid sequence at least 90% identical to the amino acid sequence given in SEQ ID NO: 1, and comprising at least one codon substitution at positions encoding R2, 13, N4, F11, E12, 163, T99, T192, A199. In some embodiments, the nucleic acid encoding a luciferase or a functional fragment thereof includes at least one codon substitution encoding the amino acid changes R2S, I3N, I3S, N4T, F11L, E12D, I63T, T99P, T192S, or A199P. In some embodiments the isolated nucleic acid encoding luciferase or a functional fragment thereof comprises SEQ ID NO: 3.

It is a further objective of the present disclosure to provide one or more expression cassettes comprising the nucleic acid of the instant disclosure under the control of regulatory elements required for nucleic acid expression in the host cell, which, being integrated into the cell genome or introduced into the cell in the form of an extrachromosomal element, is/are capable of providing the expression of luciferase encoded by the nucleic acid.

It is yet a further objective of the present disclosure to provide one or more vectors comprising the nucleic acid of the instant disclosure. The present disclosure also provides one or more vector(s) for transferring a nucleic acid into a host cell comprising a nucleic acid encoding a luciferase or a functional fragment thereof, or a fusion protein of the instant disclosure.

It is still a further objective of the present disclosure to provide one or more cells, stable cell lines, transgenic organisms (for example, plants, animals, fungi, bacteria, microorganisms) comprising the luciferases or functional fragments thereof, nucleic acids, vectors or expression cassettes of the present disclosure.

It is another objective of the present disclosure to provide a cell producing luciferase or a functional fragment thereof, comprising an expression cassette further comprising the nucleic acid under the control of regulatory elements required for nucleic acid expression in the host cell, which, being integrated into the cell genome or introduced into the cell in the form of an extrachromosomal element, is capable of providing the expression of luciferase encoded by the nucleic acid in the form of an extrachromosomal element or an element integrated into the cell genome.

It is still another objective of the present disclosure to provide a transgenic organism producing luciferase or a functional fragment thereof. In some embodiments the transgenic organism is bacterium, microorganism, plant, fungus, or animal.

It is yet another objective of the present disclosure to provide a kit comprising luciferases or functional fragments thereof, nucleic acids or vectors or expression cassettes comprising the nucleic acids of the instant disclosure.

It is a further objective of the present disclosure to provide a method for labelling cells, cell structures and biomolecules using the nucleic acids and luciferases of the instant disclosure.

It is yet a further objective of the present disclosure to provide a method for labelling cells, cell structures and biomolecules, which includes the introduction of the expression cassette for a luciferase according to the instant disclosure into a cell.

It is still a further objective of the present disclosure to provide a method for producing bioluminescence in a transgenic organism, characterized in that the transgenic organism comprises a nucleic acid encoding a luciferase or a functional fragment thereof as disclosed herein. In some embodiments the transgenic organism is bacterium, microorganism, plant, fungus, or animal.

It is another objective of the present disclosure to provide a transgenic organism for producing bioluminescence, characterized in that the transgenic organism comprises a luciferase or a functional fragment thereof, and in that the transgenic organism biosynthesizes 3-hydroxyhispidin in the cells. In some embodiments the transgenic organism is bacterium, microorganism, plant, fungus, or animal. In some embodiments, the transgenic organism produces visible bioluminescence.

It is still another objective of the present disclosure to provide a method of producing a transgenic organism comprising: providing an isolated nucleic acid encoding a luciferase or a functional fragment thereof; and incorporating the isolated nucleic acid into an extrachromosomal element or in the genome of cells comprising the organism.

It is yet another objective of the present disclosure to provide includes a method of producing a luciferase or a functional fragment thereof comprising integrating a nucleic acid encoding the luciferase or functional fragment into a genome or an extrachromosomal element (e.g., mitochondrial or plastid DNA) of a host cell, which, being provided with the necessary genetic regulatory elements, is capable of providing the expression of luciferase encoded by the nucleic acid.

It is a further objective of the present disclosure to provide transgenic bioluminescent cells and organisms capable of autonomous bioluminescence without exogenous addition of luciferin.

Hispidin hydroxylases of the instant disclosure catalyze the conversion of 6-(2-arylvinyl)-4-hydroxy-2H-pyran-2-one with the structural formula

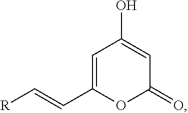

into 6-(2-arylvinyl)-3,4-dihydroxy-2H-pyran-2-one with the structural formula

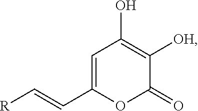

where R is aryl or heteroaryl. In some embodiments, the isolated hispidin hydroxylase or a functional fragment thereof has an amino acid sequence at least 90% identical to the amino acid sequence given in SEQ ID NO: 5 and comprises at least one amino acid substitution selected from D37E, V181I, A183P, S323M, M385K.

It is yet a further objective of the present disclosure to provide an isolated hispidin hydroxylase or a functional fragment thereof comprising at least 90%, 91%, 92%, 93%, 94%, 95%, 96%, 97%, 98%, 99% or 100% sequence identity with SEQ ID NO: 5. In some embodiments, the hispidin hydroxylase is present as a fusion to another amino acid sequence (i.e., a fusion protein).

It is still a further objective of the present disclosure to provide an amino acid sequence of the isolated hispidin hydroxylase or functional fragment thereof comprising at least one amino acid substitution at a position selected from 37, 181, 183, 323, 385 relative to SEQ ID NO: 5. Some embodiments comprise at least one substitution of the amino acids D37, V181, A183, S323, M385 relative to SEQ ID NO: 5. Some embodiments further comprise substitutions of amino acids located elsewhere within the sequence.

It is another objective of the present disclosure to provide an amino acid sequence of the isolated hispidin hydroxylase or a functional fragment thereof comprises amino acid substitutions at positions 37, 181, 323, and 385 relative to SEQ ID NO: 5. Some embodiments comprise substitutions of the amino acids D37, V181, S323, and M385 relative to SEQ ID NO: 5.

It is a further objective of the present disclosure to provide an amino acid sequence of the isolated hispidin hydroxylase, or a functional fragment thereof, that includes at least one substitution of glutamate at position 37, isoleucine at position 181, proline at position 183, methionine at position 323, or lysine at position 385 relative to SEQ ID NO: 5. In some embodiments, one or more additional amino acid substitutions may be present at other positions within the sequence.

It is yet another objective of the present disclosure to provide a hispidin hydroxylase with an amino acid sequence comprising SEQ ID NO: 6.

It is still a further objective of the present disclosure to provide one or more nucleic acid molecules encoding hispidin hydroxylases or functional fragments thereof. In some embodiments the nucleic acid molecule encodes a hispidin hydroxylase or a functional fragment thereof having an amino acid sequence at least 90% identical to the amino acid sequence given in SEQ ID NO: 5 and comprising at least one codon substitution at positions encoding D37, V181, A183, S323, M385. In some embodiments, the nucleic acid encoding a hispidin hydroxylase or a functional fragment thereof includes at least one codon substitution encoding the amino acid changes D37E, V181I, A183P, S323M, or M385K. In some embodiments the isolated nucleic acid encoding hispidin hydroxylase or a functional fragment thereof comprises SEQ ID NOs: 5.

It is a further objective of the present disclosure to provide one or more expression cassettes comprising the nucleic acid of the instant disclosure under the control of regulatory elements required for nucleic acid expression in the host cell, which, being integrated into the cell genome or introduced into the cell in the form of an extrachromosomal element, is capable of providing the expression of hispidin hydroxylase encoded by the nucleic acid.

It is yet a further objective of the present disclosure to provide one or more vectors comprising the nucleic acid as disclosed herein. The present disclosure also provides a vector for transferring a nucleic acid into a host cell comprising a nucleic acid encoding a hispidin hydroxylase or a functional fragment thereof, or a fusion protein of the instant disclosure.

It is another objective of the present disclosure to provide a fusion protein comprising operatively cross-linked, directly or via amino acid linkers, at least one a hispidin hydroxylase of the instant disclosure or a functional fragment thereof and a luciferase or a functional fragment thereof. The present disclosure also provides a nucleic acid encoding said fusion protein.

It is yet another objective of the present disclosure to provide one or more cells, stable cell lines, transgenic organisms (for example, plants, animals, fungi, bacteria, microorganisms) comprising the hispidin hydroxylase or functional fragments thereof, nucleic acids, vectors or expression cassettes of the present disclosure.

It is a further objective of the present disclosure to provide includes a cell producing hispidin hydroxylase or a functional fragment thereof according to the instant disclosure, comprising an expression cassette further comprising the nucleic acid under the control of regulatory elements required for nucleic acid expression in the host cell, which, being integrated into the cell genome or introduced into the cell in the form of an extrachromosomal element, is capable of providing the expression of hispidin hydroxylase encoded by the nucleic acid in the form of an extrachromosomal element or an element integrated into the cell genome.

It is another objective of the present disclosure to provide a transgenic organism producing hispidin hydroxylase or a functional fragment thereof. In some embodiments the transgenic organism is bacterium, microorganism, plant, fungus, or animal.

It is still another objective of the present disclosure to provide a kit comprising hispidin hydroxylase or functional fragments thereof, nucleic acids or vectors or expression cassettes comprising the nucleic acids of the instant disclosure.

It is yet another objective of the present disclosure to provide a method for labelling cells, cell structures and biomolecules using the nucleic acids and hispidin hydroxylase.

It is a further objective of the present disclosure to provide a method for labelling cells, cell structures and biomolecules, which includes the introduction of the expression cassette for a hispidin hydroxylase according to the instant disclosure into a cell.

It is yet a further objective of the present disclosure to provide a method for producing bioluminescence in a transgenic organism, characterized in that the transgenic organism comprises a nucleic acid encoding a hispidin hydroxylase or a functional fragment as disclosed herein. In some embodiments the transgenic organism is bacterium, microorganism, plant, fungus, or animal.

It is still a further objective of the present disclosure to provide a transgenic organism for producing bioluminescence, characterized in that the transgenic organism comprises a hispidin hydroxylase or a functional fragment thereof and in that the transgenic organism biosynthesizes hispidin in the cells. In some embodiments the transgenic organism is bacterium, microorganism, plant, fungus, or animal. In some embodiments, the transgenic organism produces visible bioluminescence.

It is another objective of the present disclosure to provide a method of producing a transgenic organism comprising: providing an isolated nucleic acid encoding a hispidin hydroxylase or a functional fragment thereof; and incorporating the isolated nucleic acid into an extrachromosomal element or in the genome of cells comprising the organism.

It is yet another objective of the present disclosure to provide a method of producing a hispidin hydroxylase or a functional fragment thereof, comprising integrating a nucleic acid encoding the hispidin hydroxylase or functional fragment into a genome or an extrachromosomal element (e.g., mitochondrial or plastid DNA) of a host cell, which, being provided with the necessary genetic regulatory elements, is capable of providing the expression of hispidin hydroxylase encoded by the nucleic acid.

It is still another objective of the present disclosure to provide one or more transgenic bioluminescent cells and organisms that are capable of autonomous bioluminescence without exogenous addition of hispidin.

It is a further objective of the present disclosure to provide a method for in vitro or in vivo production of 3-hydroxyhispidin by using at least one hispidin hydroxylase molecule. Methods for producing 3-hydroxyhispidin or 3-hydroxybisnoryangonin may be implemented in a cell or an organism. In one embodiment, a preferred method comprises introducing into the cell or organism nucleic acids encoding a hispidin hydroxylase of the instant disclosure and being capable of expressing said enzyme in the cell or organism. In some embodiments, the nucleic acids are introduced into a cell or organism as a part of an expression cassette or vector of the instant disclosure. In some embodiments the transgenic organism is bacterium, microorganism, plant, fungus, or animal. In preferred embodiments, the cell or organism is capable of biosynthesizing hispidin.

It is a further objective of the present disclosure to provide one or more combination(s) of proteins and/or nucleic acids as well as products and kits containing the proteins and/or nucleic acids. By way of example (but not limitation), combinations of nucleic acids are provided for producing autonomously luminous cells, cell lines, or transgenic organisms; assaying the activity of a regulatory element (i.e., promoters, enhancers, terminators, etc.) or labeling cells.

Various other objectives and advantages of the present disclosure will become apparent to those skilled in the art as a more detailed description is set forth below.

### SUMMARY OF THE INVENTION

The aforesaid and other objectives are realized by identifying one or more new variant(s) of luciferases and fungal luciferin biosynthesis enzymes, nucleic acids able to encode these enzymes, and proteins able to catalyze certain stages of the fungal luciferin biosynthesis, more specifically providing one or more bioluminescent enzymes, and in particular one or more hispidin hydroxylases and luciferases, and functional fragments thereof and nucleic acids encoding them. As described in further detail below, and illustrated throughout the various figures included herein, One or more hispidin hydroxylases of the present disclosure catalyze conversion of hispidin into the 3-hydroxyhispidin. One or more luciferases in turn oxidize 3-hydroxyhispidin resulting in emission of light.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Activity in E. coli lysates of luciferase variants obtained by consensus mutagenesis, after treatment with 100 μM of luciferin. Maximal brightness of luminescence is shown. Whiskers indicate standard deviation (N=3).

FIG. 2. Activity of nnLuz_v3 (T99P, T192S, A199P) in E. coli lysates (a) and in transient expression in HEK293 cells (b) after treatment with 100 μM luciferin. Maximal brightness of luminescence is shown. Whiskers indicate standard deviation (N=3).

FIG. 3. Stability of nnLuz variants having selected amino acid substitutions (T99P, T192S, A199P individually) in E. coli lysates after incubation at 22° C. for 23 minutes.

FIG. 4. Activity of nnLuz WT and nnLuz_v3 (T99P, T192S, A199P combined) in E. coli cleared lysates after incubation at 22° C. for different times. Data are normalized to the data at the time=0 min (N=3).

FIG. 5A. Activity of nnLuz_v3 variants having amino acid substitutions generated by random mutagenesis, in E. coli lysates (a) after treatment with luciferin. Whiskers indicate standard deviation (N=3).

FIG. 5B. Activity of nnLuz_v3 variants having amino acid substitutions generated by random mutagenesis, in transient expression in HEK293 (b) cells after treatment with luciferin. Whiskers indicate standard deviation (N=3).

FIG. 6. Activity of nnH3H variants obtained by consensus mutagenesis of nnH3H. Transient co-expression of depicted H3H mutants with nnLuz_v3 in HEK293 cells, luminescence measured after addition of 740 μM hispidin. Luminescence signal was normalized to NanoBiT assay. Red bars correspond to amino acid substitutions that provide increased luminescence. Whiskers indicate standard deviation (N=3).

FIG. 7A. Activity of nnH3H variants by transient expression in HEK293 cells after treatment with 740 μM hispidin. (a) Combinatorial analysis of amino acid substitutions in nnH3H (N=2). Whiskers indicate standard deviation (N=3).

FIG. 7B. Activity of nnH3H variants by transient expression in HEK293 cells after treatment with 740 μM hispidin. (b) Evaluation of selected combinations. Whiskers indicate standard deviation (N=3).

FIG. 8A. Insert structure of the plasmids used to create transgenic plant lines.

FIG. 8B. Insert structure of the plasmids used to create transgenic plant lines.

FIG. 9A. Comparison of bioluminescence produced in yeast (P. pastoris) by nnH3H_v2 relative to nnH3H_WT, and nnLuz_v4 relative to nnLuz_WT, by adding (a) 100 μM hispidin.

FIG. 9B. Comparison of bioluminescence produced in yeast (P. pastoris) by nnH3H_v2 relative to nnH3H_WT, and nnLuz_v4 relative to nnLuz_WT, by adding (b) 50 μM luciferin, respectively.

FIG. 10. Stability of nnLuz_v4 in yeast (P. pastoris) after 10 min incubation at different temperatures followed by treatment with luciferin (50 μM). The integral luminescence normalized to the integral signal obtained by incubation at 25° C. is shown. The color of data points indicates different yeast strains.

FIG. 11A. Comparison of various pathway versions in transient transformation assay in N. benthamiana leaves (a). Box and whisker plots are accompanied by color-coded p-values of Conover's test. NS non-significant. N=8 leaves per box plot (a). The difference between median values is indicated below the brackets between the box plots. Kruskal-Wallis H Test: H-statistic=23.59, p=3.0e-5 (a).

FIG. 11B. Comparison of various pathway versions in transient transformation assay in N. benthamiana leaves (a). Box and whisker plots are accompanied by color-coded p-values of Conover's test. NS non-significant. N=8 leaves per box plot (a). The difference between median values is indicated below the brackets between the box plots. Kruskal-Wallis H Test: H-statistic=23.59, p=3.0e-5 (a).

FIG. 11C. Comparison of various pathway versions in transient transformation assay in BY-2 cells (b). Box and whisker plots are accompanied by color-coded p-values of Conover's test. NS non-significant. N=14 cell packs per box plot (b). The difference between median values is indicated below the brackets between the box plots. Kruskal-Wallis H Test: H-statistic=48.10, p=2.0e-10 (b).

FIG. 11D. Comparison of various pathway versions in transient transformation assay in BY-2 cells (b). Box and whisker plots are accompanied by color-coded p-values of Conover's test. NS non-significant. N=14 cell packs per box plot (b). The difference between median values is indicated below the brackets between the box plots. Kruskal-Wallis H Test: H-statistic=48.10, p=2.0e-10 (b).

FIG. 12A. Average luminescence produced by leaves of 3-week-old N. benthamiana transformed with various pathway versions. The FBP1 is the plant line NB021 reported in 6. Box and whisker plots are accompanied by color-coded p-values of Conover's test. NS non-significant. The color of data points indicates different plant lines (NB021 for FBP1, 1-4 lines per box plot); N=14-68 leaves per box plot. The difference between median values is indicated below the brackets between the box plots. Kruskal-Wallis H Test: H-statistic=98.97, p=2.6e-21.

FIG. 12B. Average luminescence produced by leaves of 3-week-old N. benthamiana transformed with various pathway versions. The FBP1 is the plant line NB021 reported in 6. Box and whisker plots are accompanied by color-coded p-values of Conover's test. NS non-significant. The color of data points indicates different plant lines (NB021 for FBP1, 1-4 lines per box plot); N=14-68 leaves per box plot. The difference between median values is indicated below the brackets between the box plots. Kruskal-Wallis H Test: H-statistic=98.97, p=2.6e-21.

FIG. 13. Average luminescence produced by leaves 9-week-old N. benthamiana transformed with various pathway versions. The FBP1 is the plant line NB021 (see FIGS. 12A and 12B). The color of data points indicates different plant lines (1-4 lines per box plot); N=1-8 plants per box plot. The difference between median values is indicated below the brackets between the box plots.

FIG. 14. Comparison of various pathway versions by transient expression in mammalian cells (HEK293T). Luminescence was initiated by addition of caffeic acid. The difference between median values is indicated below the brackets between the box plots.

FIG. 15. Comparison of various pathway versions by expression in yeast cells (P. pastoris). Luminescence was initiated by addition of 100 mM caffeic acid. The difference between median values is indicated below the brackets between the box plots.

FIG. 16. Comparison of pathway versions in transient transformation assay in BY-2 cells. The difference between median values is indicated below the brackets between the box plots.

FIG. 17. Michaelis-Menten curve of nnLuz_WT and nnLuz_v4 fused with HiBiT obtained from yeast lysates in the presence of 0.1-25 μM fungal luciferin. Data normalized to expression level assessed by HiBiT luminescence. Mean values are shown with error bars representing standard deviation (N=2-3).

FIG. 18A. Activity of nnLuz mutantsin E. coli lysates, after treatment with 100 μM of luciferin. Maximal brightness of luminescence is shown. Box and whisker plots (left) are accompanied by color-coded p-values of post-hoc two-sided Conover's test (right) corrected by the step-down method using Sidak adjustments. NS non-significant. The boxes are the first and the third quartiles, whiskers are the rest of the distribution except outliers, the orange line is the median. N=3 biologically independent samples per box plot.

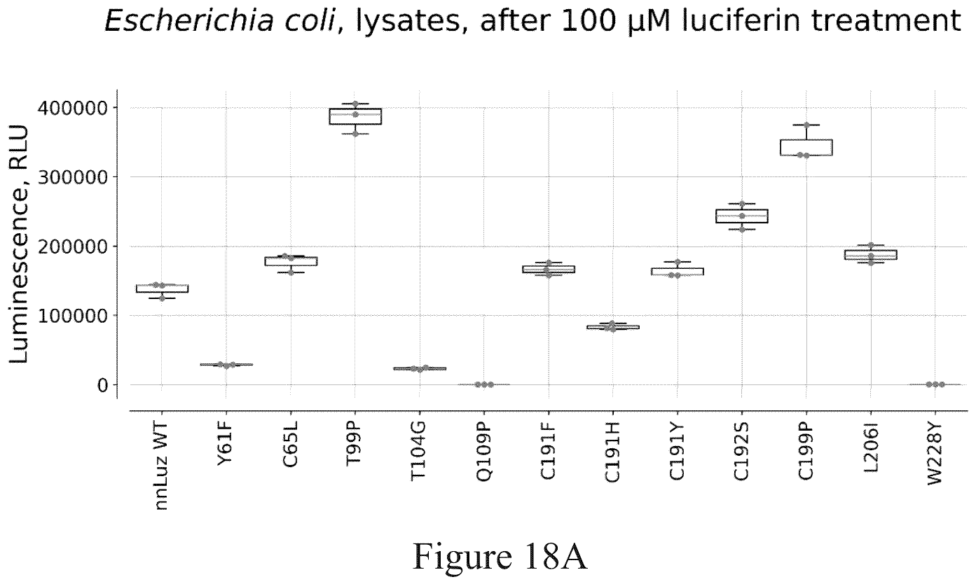

FIG. 18B. Activity of nnLuz mutantsin E. coli lysates, after treatment with 100 μM of luciferin. Maximal brightness of luminescence is shown. Box and whisker plots (left) are accompanied by color-coded p-values of post-hoc two-sided Conover's test (right) corrected by the step-down method using Sidak adjustments. NS non-significant. The boxes are the first and the third quartiles, whiskers are the rest of the distribution except outliers, the orange line is the median. N=3 biologically independent samples per box plot.

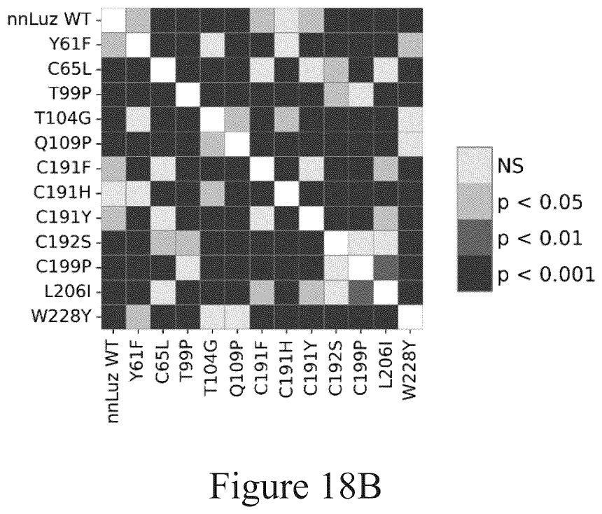

FIG. 19A. Activity of nnLuz v3 (T99P, T192S, A199P) in E. coli lysates (a) and in transient expression in HEK293 cells (b) after treatment with luciferin, respectively. Experiments in E. coli lysates were performed at room temperature, in mammalian cells—at 37° C. Maximal brightness of luminescence is shown. The boxes are the first and the third quartiles, whiskers are the rest of the distribution except outliers, the orange line is the median. The difference between mean values and p-values of post-hoc two-sided Mann-Whitney U-tests (if applicable) are indicated below the brackets between the box plots. N=4 (a) or 3 (b) biologically independent samples per box plot.

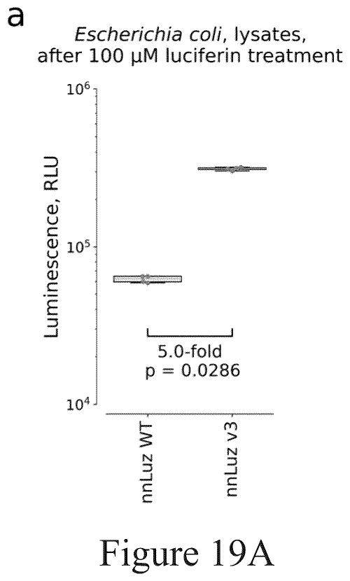

FIG. 19B. Activity of nnLuz v3 (T99P, T192S, A199P) in E. coli lysates (a) and in transient expression in HEK293 cells (b) after treatment with luciferin, respectively. Experiments in E. coli lysates were performed at room temperature, in mammalian cells—at 37° C. Maximal brightness of luminescence is shown. The boxes are the first and the third quartiles, whiskers are the rest of the distribution except outliers, the orange line is the median. The difference between mean values and p-values of post-hoc two-sided Mann-Whitney U-tests (if applicable) are indicated below the brackets between the box plots. N=4 (a) or 3 (b) biologically independent samples per box plot.

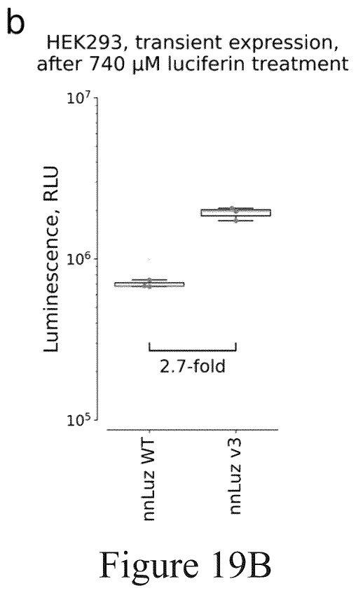

FIG. 20A. Activity of mutants obtained after random mutagenesis of nnLuz v3 in E. coli lysates (a) and in transient expression in HEK293 cells (b) after treatment with luciferin. Maximal brightness of luminescence is shown. In HEK293 luminescence signal was normalised to protein abundance, as measured by HiBiT assay. Box and whisker plots (left) are accompanied by color-coded p-values of post-hoc two-sided Conover's test (right) corrected by the step-down method using Sidak adjustments. NS non-significant. The boxes are the first and the third quartiles, whiskers are the rest of the distribution except outliers, the orange line is the median. N=3 biologically independent samples per box plot. Kruskal-Wallis H Test: H-statistic=16.58, p=5.4e-03 (a) and H-statistic=12.84, p=2.5e-02 (b).

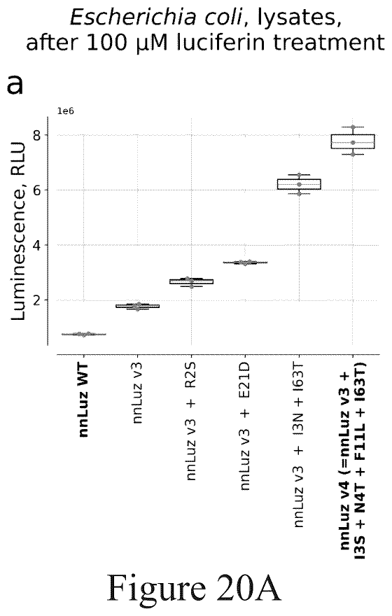

FIG. 20B. Activity of mutants obtained after random mutagenesis of nnLuz v3 in E. coli lysates (a) and in transient expression in HEK293 cells (b) after treatment with luciferin. Maximal brightness of luminescence is shown. In HEK293 luminescence signal was normalised to protein abundance, as measured by HiBiT assay. Box and whisker plots (left) are accompanied by color-coded p-values of post-hoc two-sided Conover's test (right) corrected by the step-down method using Sidak adjustments. NS non-significant. The boxes are the first and the third quartiles, whiskers are the rest of the distribution except outliers, the orange line is the median. N=3 biologically independent samples per box plot. Kruskal-Wallis H Test: H-statistic=16.58, p=5.4e-03 (a) and H-statistic=12.84, p=2.5e-02 (b).

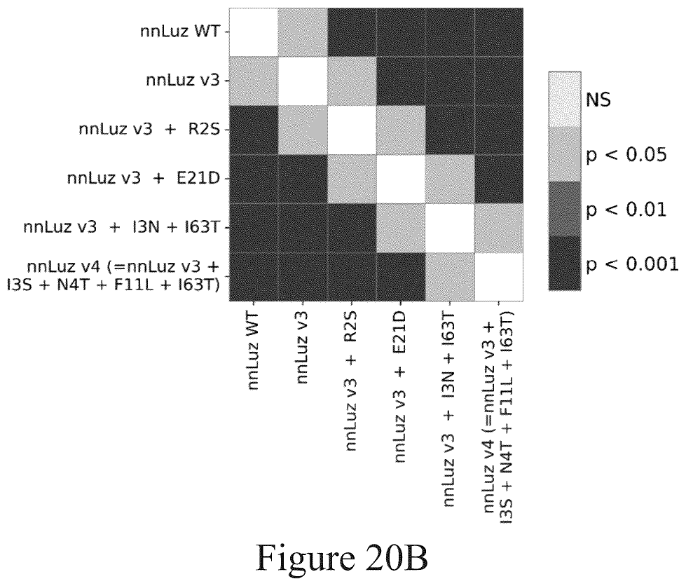

FIG. 20C. Activity of mutants obtained after random mutagenesis of nnLuz v3 in E. coli lysates (a) and in transient expression in HEK293 cells (b) after treatment with luciferin. Maximal brightness of luminescence is shown. In HEK293 luminescence signal was normalised to protein abundance, as measured by HiBiT assay. Box and whisker plots (left) are accompanied by color-coded p-values of post-hoc two-sided Conover's test (right) corrected by the step-down method using Sidak adjustments. NS non-significant. The boxes are the first and the third quartiles, whiskers are the rest of the distribution except outliers, the orange line is the median. N=3 biologically independent samples per box plot. Kruskal-Wallis H Test: H-statistic=16.58, p=5.4e-03 (a) and H-statistic=12.84, p=2.5e-02 (b).

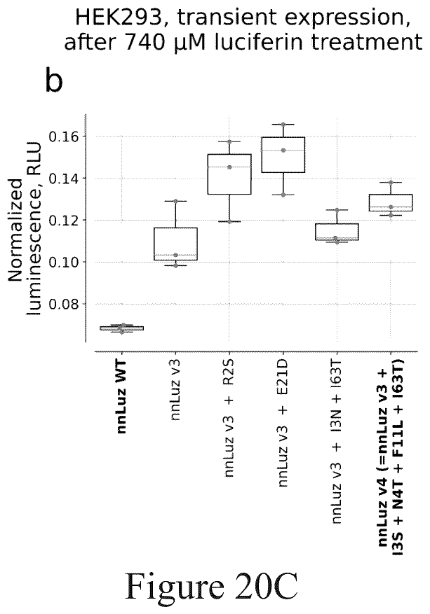

FIG. 20D. Activity of mutants obtained after random mutagenesis of nnLuz v3 in E. coli lysates (a) and in transient expression in HEK293 cells (b) after treatment with luciferin. Maximal brightness of luminescence is shown. In HEK293 luminescence signal was normalised to protein abundance, as measured by HiBiT assay. Box and whisker plots (left) are accompanied by color-coded p-values of post-hoc two-sided Conover's test (right) corrected by the step-down method using Sidak adjustments. NS non-significant. The boxes are the first and the third quartiles, whiskers are the rest of the distribution except outliers, the orange line is the median. N=3 biologically independent samples per box plot. Kruskal-Wallis H Test: H-statistic=16.58, p=5.4e-03 (a) and H-statistic=12.84, p=2.5e-02 (b).

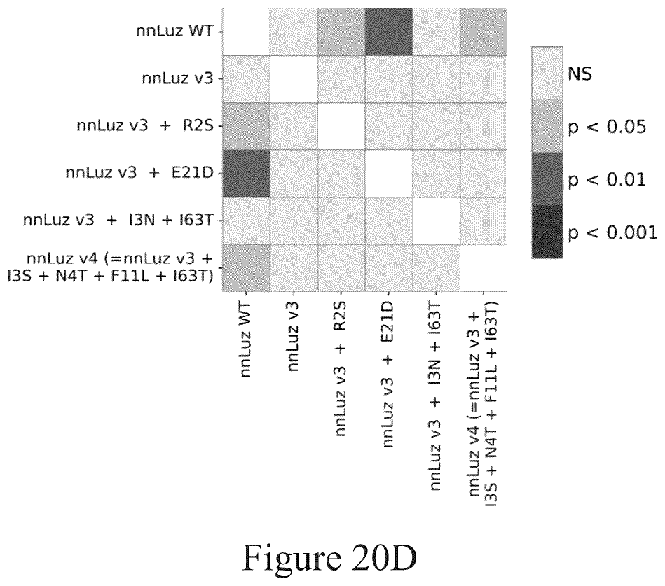

FIG. 21A. Activity of mutants of nnH3H. Transient co-expression of depicted nnH3H mutants with nnLuz_v3 in HEK293 cells, luminescence measured after addition of 0.8 mM hispidin. Luminescence signal was normalised to protein abundance, as measured by HiBiT assay. Red labels correspond to mutations with improved luminescence. Box and whisker plots (left) are accompanied by color-coded p-values of post-hoc two-sided Conover's test (right) corrected by the step-down method using Sidak adjustments. NS non-significant. The boxes are the first and the third quartiles, whiskers are the rest of the distribution except outliers, the orange line is the median. N=3 biologically independent samples per box plot. Kruskal-Wallis H Test: H-statistic=82.22, p=3.1e-07.

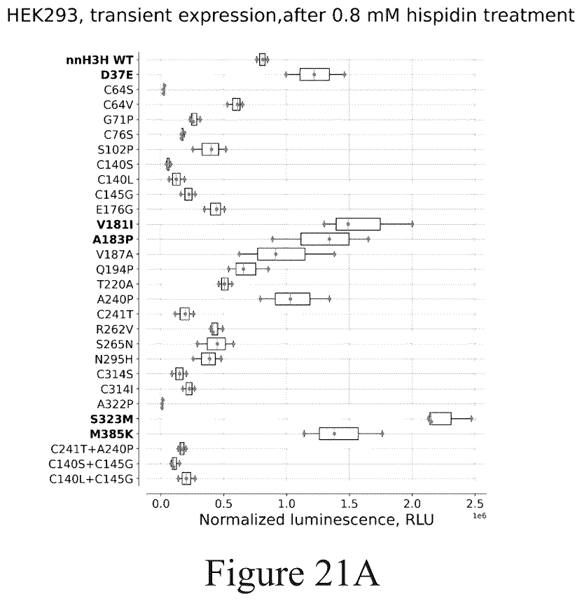

FIG. 21B. Activity of mutants of nnH3H. Transient co-expression of depicted nnH3H mutants with nnLuz_v3 in HEK293 cells, luminescence measured after addition of 0.8 mM hispidin. Luminescence signal was normalised to protein abundance, as measured by HiBiT assay. Red labels correspond to mutations with improved luminescence. Box and whisker plots (left) are accompanied by color-coded p-values of post-hoc two-sided Conover's test (right) corrected by the step-down method using Sidak adjustments. NS non-significant. The boxes are the first and the third quartiles, whiskers are the rest of the distribution except outliers, the orange line is the median. N=3 biologically independent samples per box plot. Kruskal-Wallis H Test: H-statistic=82.22, p=3.1e-07.

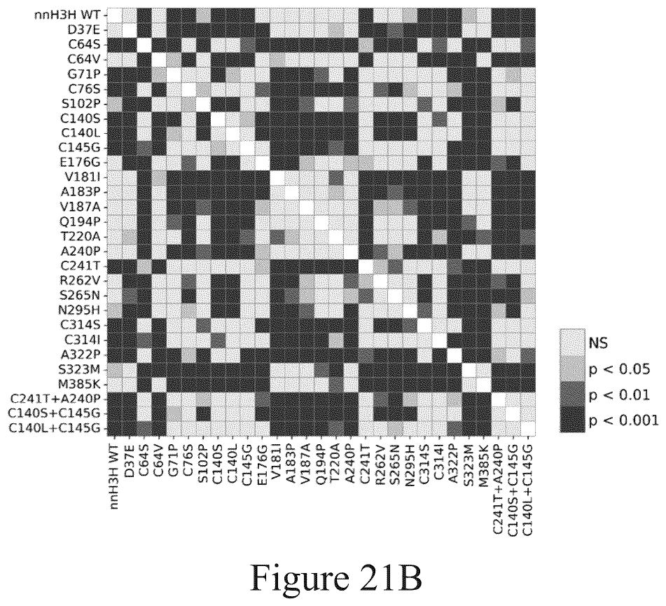

FIG. 22A. Activity of nnH3H mutants in transient expression with nnLuz_v3 in HEK293 after treatment with 0.8 mM of luciferin or hispidin. a. Combinatorial mutation analysis of nnH3H. N=2 biologically independent samples per box plot. b. Evaluation of selected combinations. N=3 biologically independent samples per box plot. The boxes are the first and the third quartiles, whiskers are the rest of the distribution except outliers, the orange line is the median.

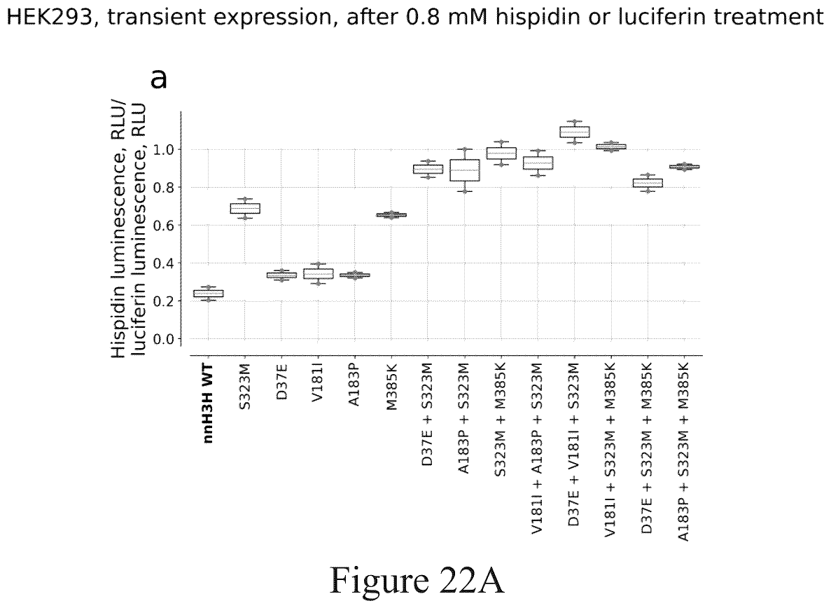

FIG. 22B. Activity of nnH3H mutants in transient expression with nnLuz_v3 in HEK293 after treatment with 0.8 mM of luciferin or hispidin. a. Combinatorial mutation analysis of nnH3H. N=2 biologically independent samples per box plot. b. Evaluation of selected combinations. N=3 biologically independent samples per box plot. The boxes are the first and the third quartiles, whiskers are the rest of the distribution except outliers, the orange line is the median.

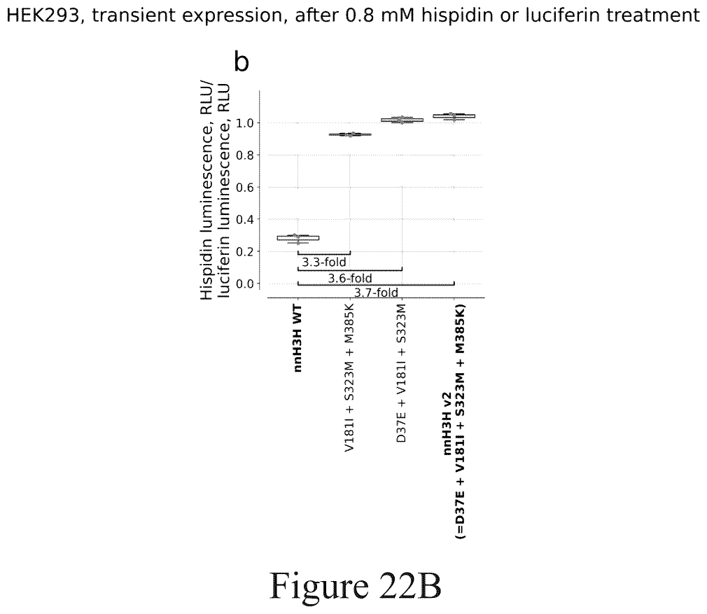

FIG. 23A. Comparison of Luz_v4 and Luz_v5 in BY-2 cells. Different optical densities of suspensions (OD600) of agrobacterial strains encoding the luciferase genes are designated in the figure labels. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments. NS non-significant.

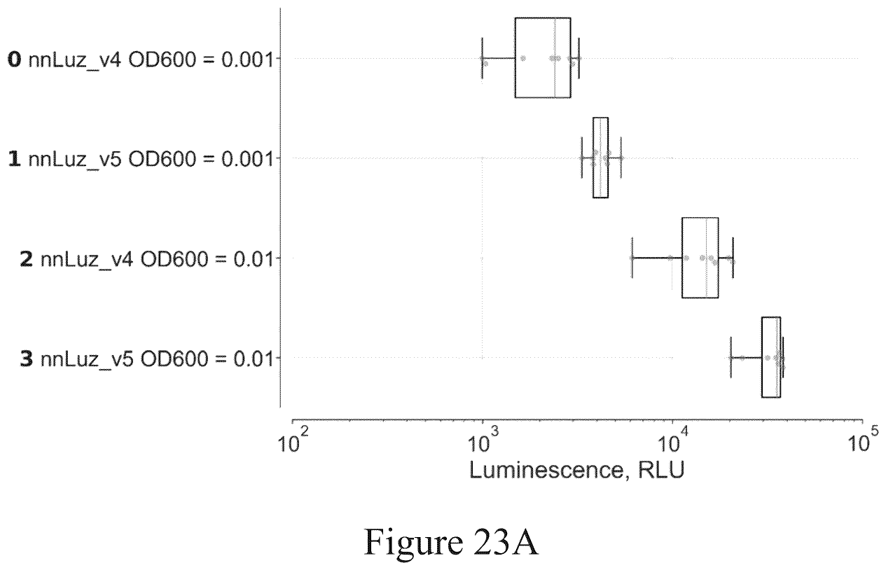

FIG. 23B. Comparison of Luz_v4 and Luz_v5 in BY-2 cells. Different optical densities of suspensions (OD600) of agrobacterial strains encoding the luciferase genes are designated in the figure labels. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments. NS non-significant.

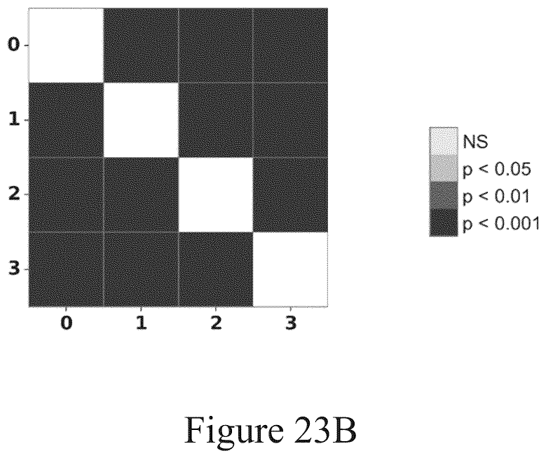

FIG. 24A. Comparison of H3H_v2 and H3H_v4 in BY-2 cells. Different optical densities of suspensions (OD600) of agrobacterial strains encoding the hispidin-3-hydroxylases genes are designated in the figure labels. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments. NS non-significant.

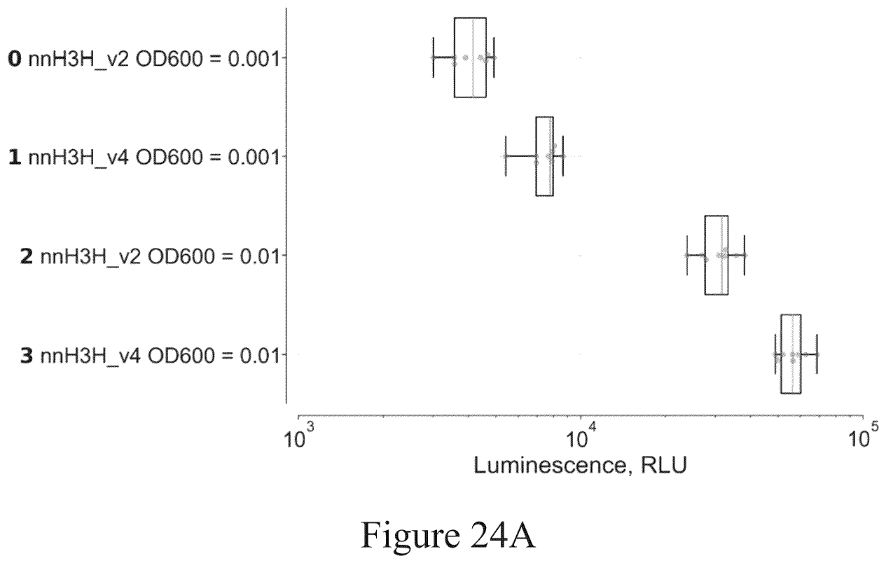

FIG. 24B. Comparison of H3H_v2 and H3H_v4 in BY-2 cells. Different optical densities of suspensions (OD600) of agrobacterial strains encoding the hispidin-3-hydroxylases genes are designated in the figure labels. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments. NS non-significant.

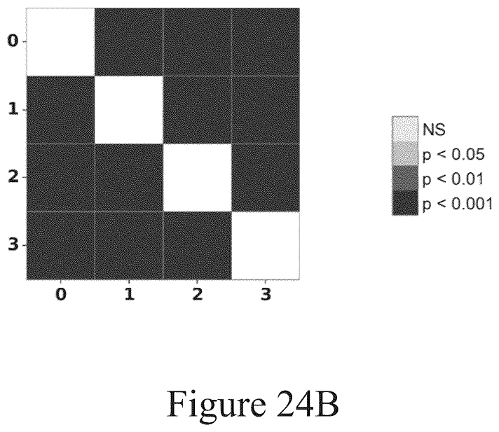

FIG. 25A. Comparison of Luz_v5 and H3H_v4 in fusion and as separate enzymes in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments. NS non-significant.

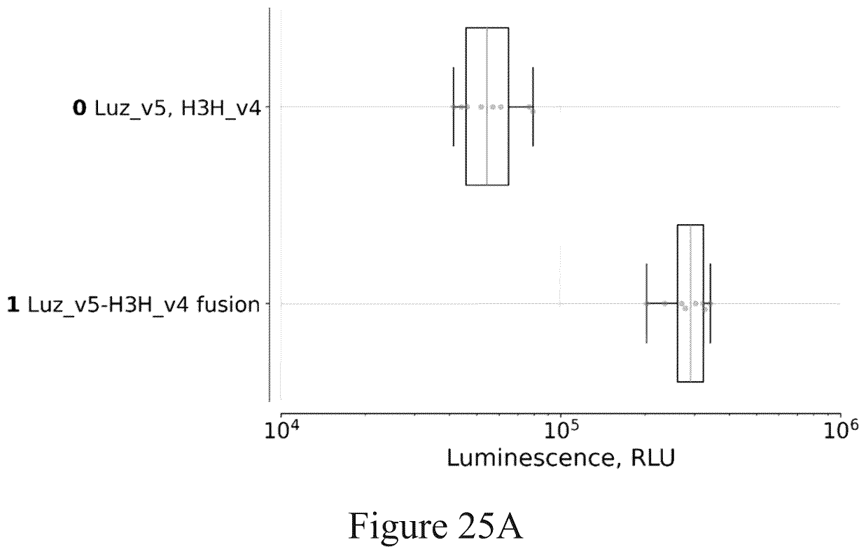

FIG. 25B. Comparison of Luz_v5 and H3H_v4 in fusion and as separate enzymes in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments. NS non-significant.

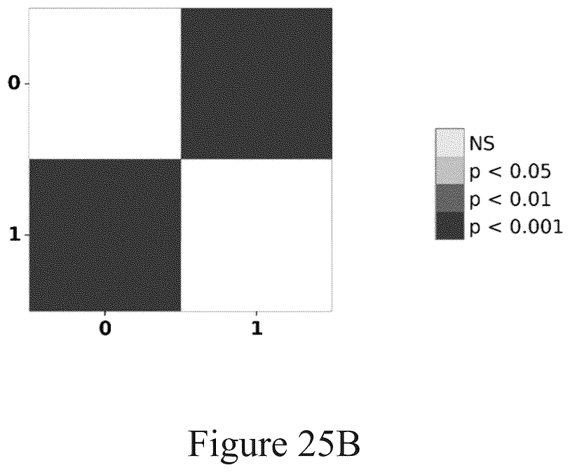

FIG. 26A. Multiple sequence alignment of amino acid sequences of luciferases. Differences are highlighted by shades of varying depth. Positions selected for experimental validation are designated by asterisk.

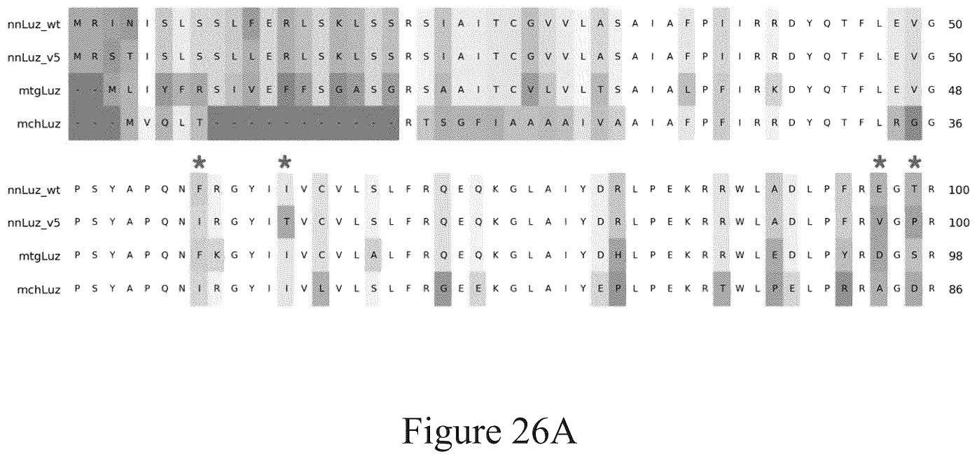

FIG. 26B. Multiple sequence alignment of amino acid sequences of luciferases. Differences are highlighted by shades of varying depth. Positions selected for experimental validation are designated by asterisk.

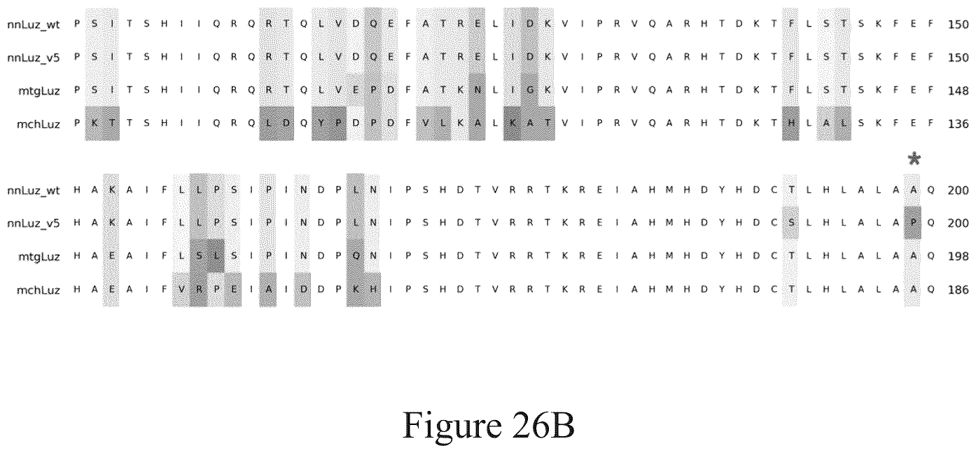

FIG. 26C. Multiple sequence alignment of amino acid sequences of luciferases. Differences are highlighted by shades of varying depth. Positions selected for experimental validation are designated by asterisk.

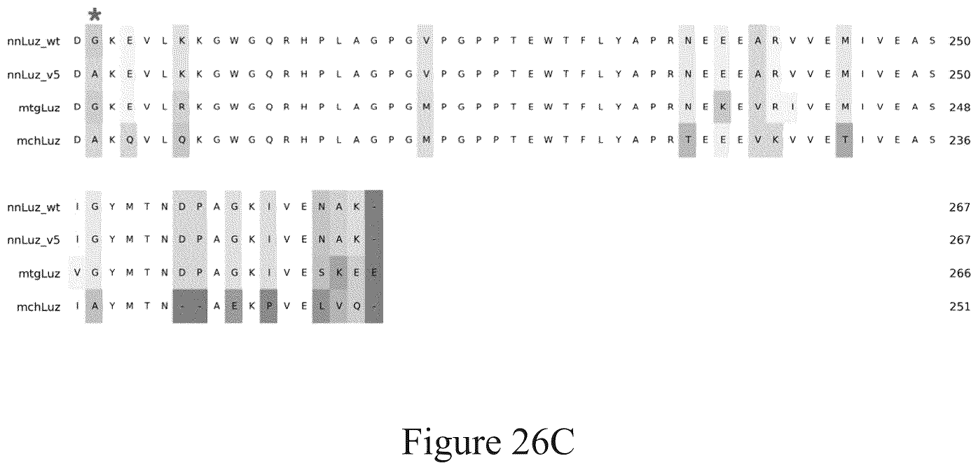

FIG. 27A. Measuring the effect of selected amino acid substitutions in the context of sequence of luciferase mtgLuz in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments.

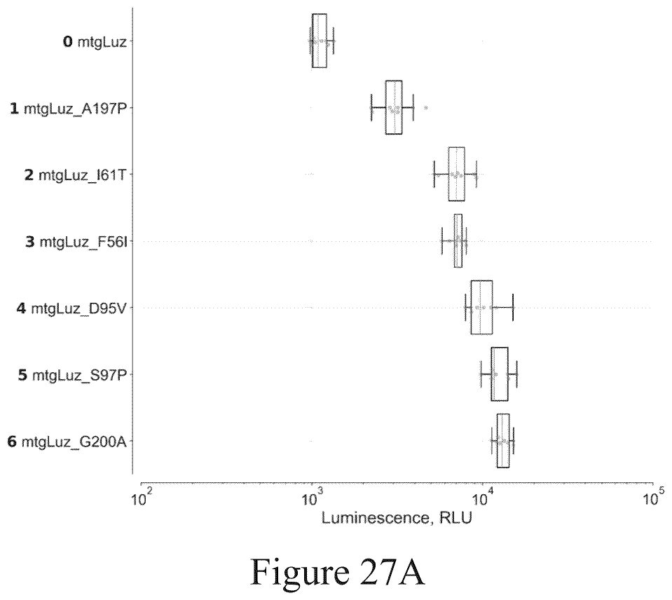

FIG. 27B. Measuring the effect of selected amino acid substitutions in the context of sequence of luciferase mtgLuz in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments.

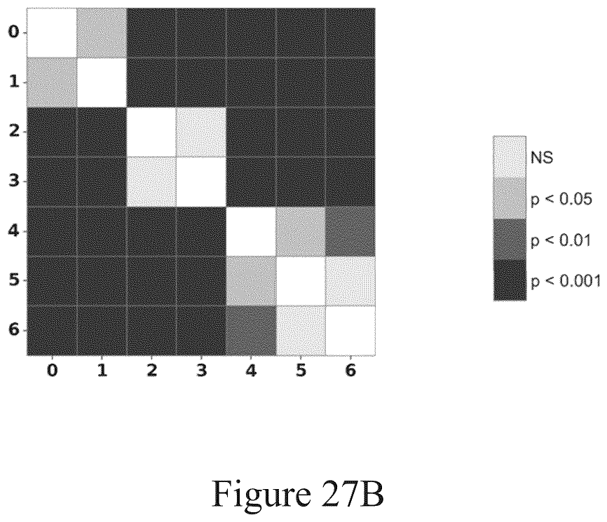

FIG. 28A. Measuring the effect of selected amino acid substitutions in the context of sequence of luciferase mchLuz in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments.

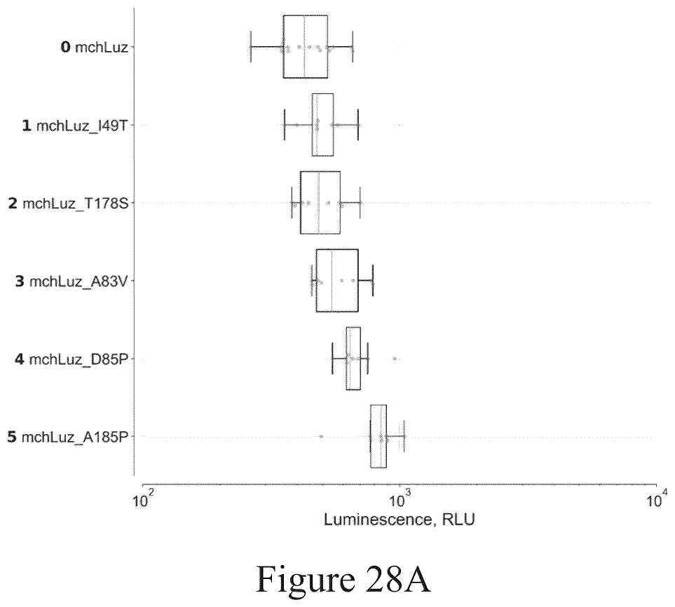

FIG. 28B. Measuring the effect of selected amino acid substitutions in the context of sequence of luciferase mchLuz in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments.

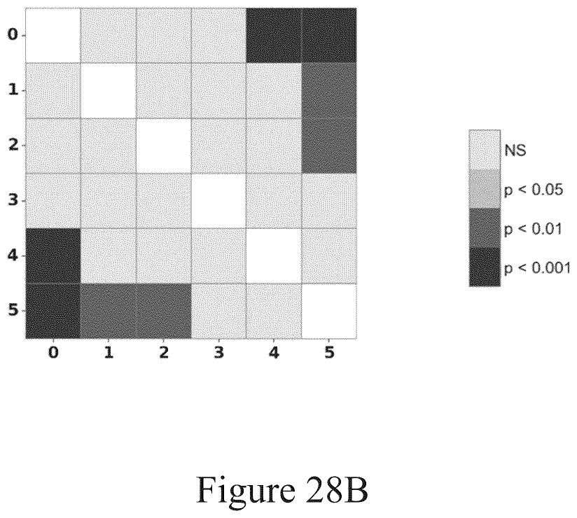

FIG. 29A. Measuring the effect of selected amino acid substitutions in the context of sequence of hispidin-3-hydroxylase aostH3H in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments.

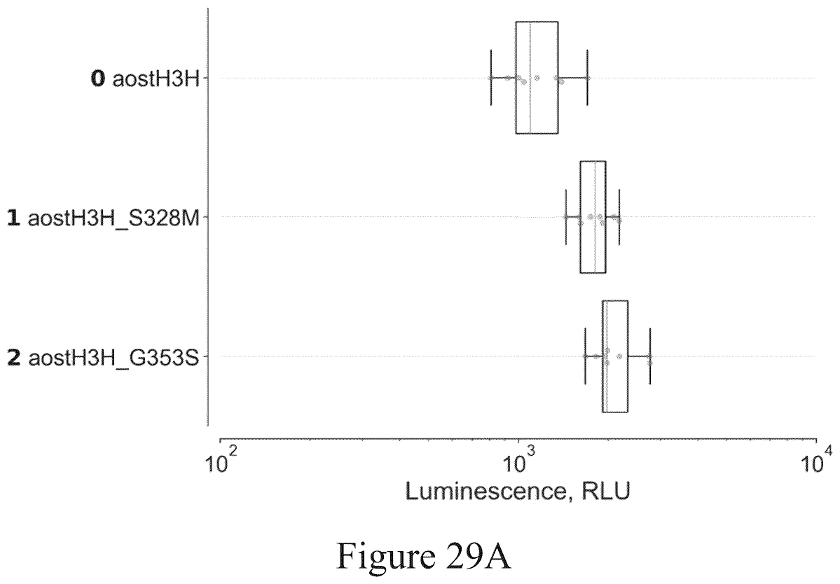

FIG. 29B. Measuring the effect of selected amino acid substitutions in the context of sequence of hispidin-3-hydroxylase aostH3H in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments.

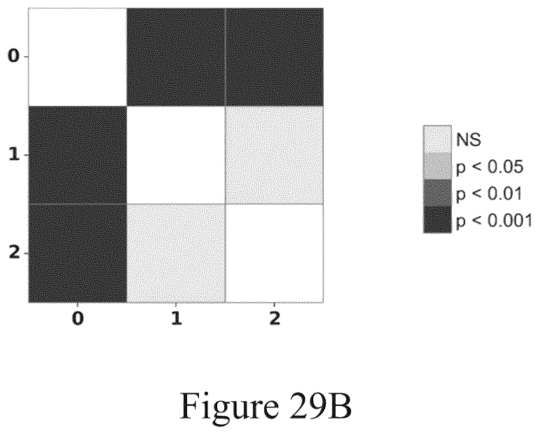

FIG. 30A. Comparison of mutants of mtgLuz-aostH3H fusion in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments.

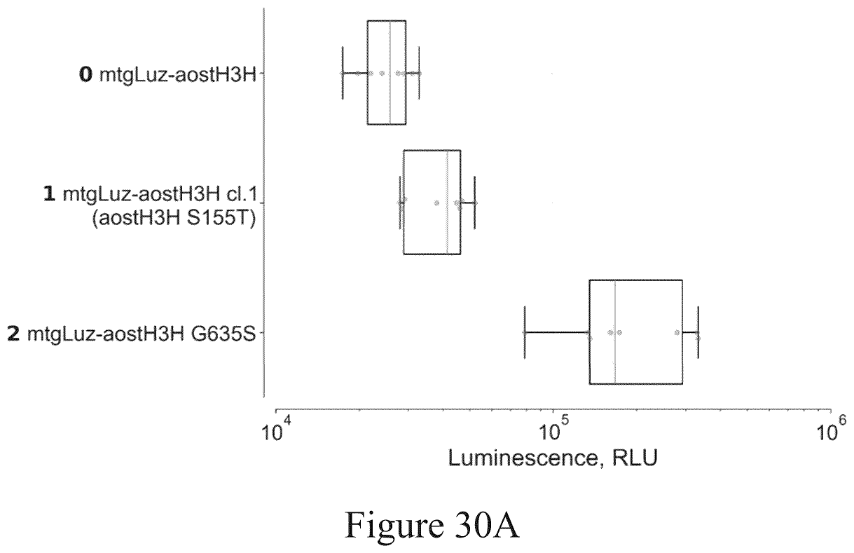

FIG. 30B. Comparison of mutants of mtgLuz-aostH3H fusion in BY-2 cells. The boxes are the first and the third quartiles, whiskers are the rest of the distribution except outliers, the orange line is the median. N=8 per boxplot. Box and whisker plots are accompanied by color-coded p-values of two-sided Conover's test corrected by the step-down method using Sidak adjustments.

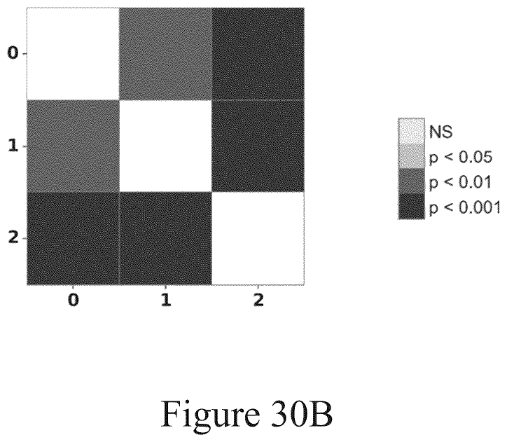

### DETAILED DESCRIPTION OF THE PREFERRED EMBODIMENT AND OPERATION OF THE INVENTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

Various exemplary embodiments of the present disclosure are described below. Use of the term “exemplary” means illustrative or by way of example only, and any reference herein to “the disclosure” is not intended to restrict or limit the disclosure to exact features or step of any one or more of the exemplary embodiments disclosed in the present specification. References to “exemplary embodiment”, “one embodiment”, “an embodiment”, “various embodiments”, and the like may indicate that the embodiment(s) of the disclosure so described may include a particular feature, structure, or characteristic, but not every embodiment necessarily incudes the particular feature, structure, or characteristic. Further, repeated use of the phrase “in one embodiment”, “in an exemplary embodiment”, or “in an alternative embodiment” do not necessarily refer to the same embodiment, although they may.

It is also noted that terms like “preferably”, “commonly”, and “typically” are not utilized herein to limit the scope of the disclosure or to imply that certain features are critical, essential, or even important to the structure or function of the disclosure. Rather, these terms are merely intended to highlight alternative or additional features that may or may not be utilized in a particular embodiment of the present disclosure.

The present disclosure is described more fully hereinafter with reference to the accompanying figures, in which one or more exemplary embodiments of the disclosure are shown. Like numbers used herein refer to like elements throughout. The disclosure may, however, be embodied in many different forms and should not be construed as limited to the embodiments set forth herein; rather, these embodiments are provided so that this disclosure will be operative, enabling, and complete. Accordingly, the particular arrangements disclosed are meant to be illustrative only and not limited as to the scope of the disclosure, and any and all equivalents thereof. Moreover, many embodiments such as adaptations, variations, modifications, and equivalent arrangements will be implicitly disclosed by the embodiments described herein and fall within the scope of the instant disclosure.

Although specific terms are employed herein, they are used in a generic and descriptive sense only and not for the purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. Where only one item is intended, the terms “one and only one”, “single”, or similar language is used. When used herein to join a list of items, the term “or” denotes at least one of the items but does not exclude a plurality of items of the list. The terms “comprise(s),” “include(s),” “having,” “has,” “can,” “contain(s),” and variants thereof, as used herein, are intended to be open-ended transitional phrases, terms, or words that do not preclude the possibility of additional acts or structures. The terminology used herein is for the purpose of describing particular embodiments only and is not intended to be limiting. The present disclosure also contemplates other embodiments “comprising,” “consisting of” and “consisting essentially of,” the embodiments or elements presented herein, whether explicitly set forth or not.

For exemplary methods or processes of the disclosure, the sequence and/or arrangement of steps described herein are illustrative and not restrictive. Accordingly, it should be understood that, although steps of various processes or methods may be shown and described as being in a sequence or temporal arrangement, the steps of any such processes or methods are not limited to being carried out in any particular sequence or arrangement, absent an indication otherwise. Indeed, the steps in such processes or methods generally may be carried out in various different sequences and arrangements while still falling within the scope of the present disclosure.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Additionally, any references to advantages, benefits, unexpected results, or operability of the present disclosure are not intended as an affirmation that the disclosure has previously been reduced to practice or that any testing has been performed. Likewise, unless stated otherwise, use of verbs in the past tense (present perfect or preterit) is not intended to indicate or imply that the disclosure has previously been reduced to practice or that any testing has been performed.

Names of chemical compounds are used in the present disclosure in accordance with the international IUPAC nomenclature. Traditional names are presented as well (if any).

The term or phrase “luciferin biosynthesizing enzyme”, or “enzyme involved in cyclic turnover of luciferin conversions”, or the like is used to mean an enzyme that catalyzes the conversion of a preluciferin precursor to preluciferin, and/or preluciferin to fungal luciferin, and/or oxyluciferin to a preluciferin precursor, in in vitro and/or in vivo systems. The term “fungal luciferin biosynthesizing enzyme” does not cover luciferases, unless otherwise specified.

As used herein, the term “luciferase” means a protein capable of oxidizing luciferin where the oxidation reaction is accompanied by the emission of light (luminescence) and oxidized luciferin is released.

The terms “luminescence” and “bioluminescence” are interchangeable for the purposes of present disclosure and refer to the phenomenon of light emission in course of a chemical reaction catalyzed by the enzyme luciferase. The luciferase derived from Neonothopanus nambi is denoted as nnLuz.

The term “hispidin hydroxylase” is used herein to describe the enzyme that catalyzes reaction of converting preluciferin to fungal luciferin, for example, synthesizing 3-hydroxyhispidin from hispidin. A preferred hispidin hydroxylase may be derived from fungi, and typically also synthesizes 3-hydroxybisnoryangonin from bisnoryangonin. The hispidin hydroxylase derived from Neonothopanus nambi is denoted as nnH3H herein.

The term “hispidin synthase” is used herein to describe an enzyme capable to catalyze synthesis of fungal preluciferin from a precursor of preluciferin, for example, synthesis of hispidin from caffeic acid. The hispidin synthase derived from Neonothopanus nambi is denoted as nnHispS. The hispidin synthase derived from Mycena citricolor is denoted as mcitHispS.

The term “caffeylpyruvate hydrolase” is used herein to describe an enzyme capable to catalyze decomposition of fungal oxyluciferin into simpler compounds, for example, to form a precursor of preluciferin. For example, it can catalyze conversion of caffeylpyruvate to caffeic acid. The caffeylpyruvate hydrolase derived from Neonothopanus nambi is denoted as nnCPH.

The term “phosphopantetheinyl transferase” is used to describe an enzyme capable to catalyze the transfer of phosphopantetheine to another protein, for example a hispidin synthase. Phosphopantetheine, also known as 4′-phosphopantetheine, is a prosthetic group of several acyl carrier proteins including the acyl carrier proteins (ACP) of fatty acid synthases, ACPs of polyketide synthases, the peptidyl carrier proteins (PCP), as well as aryl carrier proteins (ArCP) of nonribosomal peptide synthetases (NRPS). A phosphopantetheinyl transferase is denoted as PPTase. A phosphopantetheinyl transferase derived from is denoted as Aspergillus nidulans is is denoted NpgA.

The term or phrase “functional analogue” is used in the present disclosure to describe chemical compounds or proteins that perform the same function and/or can be used for the same purpose.

The term “ATP” refers to adenosine triphosphate, which is the main carrier of energy in the cell and has the structural formula:

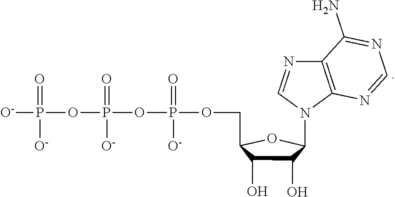

The term “NAD(P)H” is used herein to refer to the reduced nicotinamide adenine dinucleotide phosphate (NADPH) moiety or nicotinamide adenine dinucleotide (NADH) moiety. Term “NAD(P)” is used to refer to the oxidized form of nicotinamide adenine dinucleotide phosphate (NADP) or nicotinamide adenine dinucleotide (NAD). Nicotinamide adenine dinucleotide:

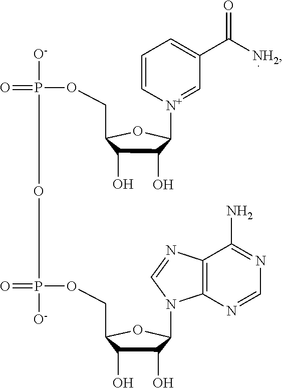

and nicotinamide adenine dinucleotide phosphate:

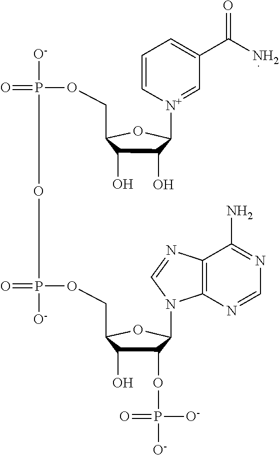

are dinucleotides built from nicotinic acid amide and adenine linked by a chain consisting of two D-ribose residues and two phosphoric acid residues. NADP differs from NAD by presence of additional phosphoric acid residue attached to hydroxyl of a D-ribose residue. Both compounds are widespread in nature and participate in many redox reactions, performing function of carriers of electrons and hydrogen, which it receives from oxidized substances. The reduced forms transfer the received electrons and hydrogen to other substances.

The term “coenzyme A” or “CoA” refers to a coenzyme well known from the prior art, which is involved in oxidation or synthesis of fatty acids, biosynthesis of fats, oxidative transformations of carbohydrate decomposition products and has the structural formula:

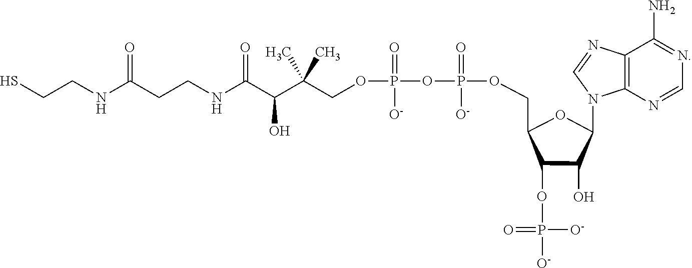

The term “malonyl-CoA” refers to a derivative of coenzyme A formed during synthesis of fatty acids and containing a malonic acid residue:

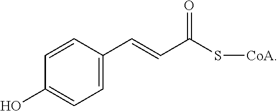

The term “coumaroyl-CoA” refers to the thioester of coenzyme A and coumaric acid:

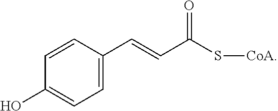

The term “caffeyl-CoA” refers to the thioester of coenzyme A and caffeic acid:

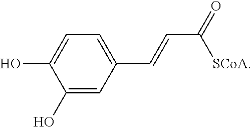

The term “mutant” or “derivative”, as used herein, refers to a protein disclosed in the present disclosure, wherein one or more amino acids are added to, and/or substituted at, and/or removed (deleted) from, and/or incorporated (inserted) into N-terminus, and/or C-terminus, and/or a native amino acid sequence within a protein of the present disclosure. As used here, the term “mutant” refers to a nucleic acid moiety that encodes a mutant protein. Besides, the term “mutant”, as used herein, refers to any variant that is shorter or longer than the protein or nucleic acid disclosed in the present disclosure.

For the purposes of present disclosure, term “fungal luciferin” is 3-hydroxyhispidin, term “pre-luciferin (precursor)” is hispidin. Oxidation of fungal luciferin produces a “fungal oxyluciferin”.

The term or phrase “luciferin biosynthesizing enzyme”, or “enzyme involved in cyclic turnover of luciferin conversions”, or the like is used to mean an enzyme that catalyzes the conversion of a preluciferin precursor to preluciferin, and/or preluciferin to fungal luciferin, and/or oxyluciferin to a preluciferin precursor, in in vitro and/or in vivo systems. The term “fungal luciferin biosynthesizing enzyme” does not cover luciferases, unless otherwise specified.

The term “amino acid insertion segment” means one or more amino acids within a polypeptide chain that are between protein fragments (protein domains, linkers, consensus sequences) under consideration. It should be obvious to those skilled in the art that the amino acid insertion segments and fragments under consideration are operatively linked and form a single polypeptide chain.

The domain structure of a protein may be determined using any suitable software known in the art. For example, a Simple Modular Architecture Research Tool (SMART) software available in Internet at http://smart.embl-heidelberg.de can be used for this purpose [see for example Schultz et al., PNAS 1998; 95: 5857-5864; Letunic I, Doerks T, Bork P, Nucleic Acids Res 2014; doi:10.1093/nar/gku949].

The term “operatively linked” or the like in description of fusion proteins refers to polypeptide sequences that occur in a physical and functional relationship with one another. In most preferred embodiments, functions of polypeptide components of the chimeric molecule are not altered as compared with functional properties of the unlinked polypeptide components. For example, the hispidin hydroxylase of the present disclosure can be operatively linked to a fusion partner of interest, e.g. luciferase. In this case, the fusion protein retains the properties of hispidin hydroxylase while the polypeptide of interest retains its original biological activity, for example, the ability to oxidize luciferin with light emission. In some embodiments of the present invention, activities of the fusion partners may be reduced compared with activities of the unlinked proteins. Such fusion proteins also find application within the scope of the present invention.

The term “operatively linked” or the like in description of nucleic acids means that the nucleic acids are covalently linked in such a way that there are no reading frame malfunctions or stop signs at their junctions. As it is obvious to any person skilled in the art, nucleotide sequences encoding a fusion protein with the “operatively linked” components (proteins, polypeptides, linker sequences, amino acid insertion segments, protein domains, etc.) are composed of fragments encoding said components, these fragments being covalently linked in such a way that a full-length fusion protein is produced during transcription and translation of the nucleotide sequence.

The term “operatively linked” in description of a nucleic acid relationship with regulatory coding sequences (promoters, enhancers, transcription terminators) means that the sequences are located and linked in such a way that the regulatory sequence will affect the expression level of the coding nucleic acid or nucleic acid sequence.

In the context of the present invention, “linking” of nucleic acids means that two or more nucleic acids are linked together using any means known in the art. As a non-limiting example, nucleic acids can be linked together using DNA ligase or polymerase chain reaction (PCR) during annealing. Nucleic acids can also be linked by chemical synthesis of a nucleic acid using a sequence of two or more separate nucleic acids.

The term or phrase “regulatory elements” or “regulatory sequences” refer to the sequences involved in a coding nucleic acid expression regulation. Regulatory elements include promoters, termination signals, and other sequences that affect the expression of a nucleic acid. They typically also comprise the sequences required for proper translation of the nucleotide sequence.

The term “promoter” is used to describe an untranslated and non-transcribed DNA sequence upstream of the coding region that contains a RNA polymerase binding site as well as transcription initiating DNA binding site. Promoter region can also comprise another gene expression regulating elements.

The term “functional”, as used here, refers to a nucleotide or amino acid sequence that can play a role in a particular test or task. Term “functional”, if used to describe luciferases, means that the protein has the ability to produce the reaction of luciferin oxidation accompanied by luminescence. The same term “functional”, if used to describe hispidin hydroxylases, means that the protein has the ability to catalyze reaction of converting the preluciferin to the luciferin. The same term “functional”, if used to describe hispidin synthases, means that the protein has the ability to catalyze reaction of converting at least one of precursors of preluciferin to preluciferin, for example, converting caffeic acid to hispidin. The same term “functional”, if used to describe caffeylpyruvate hydrolases, means that the protein has the ability to catalyze reaction of converting at least one of oxyluciferins to precursor of preluciferin (for example, converting caffeylpyruvate to caffeic acid).

The term “enzymatic properties”, as used here, refers to the ability of a protein to catalyze a given chemical reaction.

The term “biochemical properties”, as used here, refers to protein folding and comprises maturation rate, half-life, catalysis rate, pH and temperature stability, and other similar properties.

As used herein, “spectral properties” refer to spectra, quantum efficiency and luminescence intensity, and other similar properties.

As used herein, the term “isolated” means a molecule or cell which is in an environment different from an environment in which the molecule or cell exist under natural conditions. In some embodiments, an isolated molecule is substantially free from other natural biological molecules such as proteins, oligosaccharides, nucleic acids and fragments thereof, etc. In this context, the term “substantially free” means that less than 70%, normally less than 60%, and more often less than 50% of said composition containing isolated molecules is made up of other natural biological molecules. In some embodiments, said isolated molecules are in substantially pure form. In this context, the term “substantially pure form” means that said molecules are at least 95% pure, normally at least 97% pure, and more often at least 99% pure.

The term “homology” is used to describe interconnection of nucleotide or amino acid sequences with other nucleotide or amino acid sequences; this interconnection is determined by the degree of identity and/or similarity between these compared sequences.

As used herein, the amino acid or nucleotide sequence is “substantially similar” or “substantially identical” to a reference sequence provided the amino acid or nucleotide sequences are at least 90% identical to the specified sequence within the region selected for comparison. Thus, substantially similar sequences are those which are, for example, at least 90% identical—for example, 90%, 91%, 92%, 93%, 94%, 95%, 96%, 97%, 98%, 98% or 99% identical. Two sequences which are identical to each other are also substantially similar. For the purposes of the present invention, the length of luciferase sequences being compared is at least 100 amino acids, preferably at least 200 amino acids, for example 200-230 amino acids or the full length of amino acid sequence of a protein or functional fragment. As for the nucleic acids, the length of the sequences being compared is commonly at least 300 nucleotides, preferably at least 600 nucleotides.

Fungal luciferases contain a highly variable transmembrane region at the N-terminus. This transmembrane region does not contribute to the structure of the catalytic domain and may be removed or replaced with other amino acid sequences. Such modifications do not significantly alter the catalytic mechanism or the spectral properties of the bioluminescence but may influence intracellular characteristics such as localization or expression half-life. Accordingly, the amino acid sequence of the catalytic domain is considered ‘substantially similar’ or ‘substantially identical’ to a reference sequence if the amino acid or nucleotide sequences are at least 90% identical to the specified catalytic region. The catalytic region of nnLuz comprises amino acids 40-267 as presented in SEQ ID NO: 1.

Identity of sequences is determined based on a reference sequence. Algorithms for sequence analysis are known in the art, for example, BLAST described in Altschul et al., J. Mol. Biol., 215, pp. 403-10 (1990). For the purposes of the present disclosure, comparison of nucleotide and amino acid sequences by way of Blast software package provided by the National Center for Biotechnology Information (http://www.ncbi.nlm.nih.gov/blast) using gapped alignment with standard parameters can be used to determine the degree of identity and similarity between the nucleotide sequences and amino acid sequences.

A reference to a nucleotide sequence “encoding” polypeptide means that such polypeptide is produced from a nucleotide sequence during mRNA transcription and translation. For this, both a coding strand, which is identical to mRNA and commonly used in the sequence listing, and a complementary strand, which is used as a template during transcription, can be specified. It will be appreciated by those having skill in the art that the term also includes any degenerate nucleotide sequences encoding a uniform amino acid sequence. Nucleotide sequences encoding polypeptide include sequences which contain introns.

The term or phrase “expression cassette” or “cassette of expression” are used herein in sense of a nucleic acid sequence capable to regulate expression of a particular nucleotide sequence in an appropriate host cell. As a rule, the “expression cassette” contains a heterologous nucleic acid encoding a protein or a functional fragment thereof operatively linked to a promoter and termination signals. Typically, it also contains sequences required for proper translation of a significant nucleotide sequence. The expression cassette may be one that occurs in nature (including host cells), but has been produced in a recombinant form useful for expression of the heterologous nucleic acid. However, in many cases, the “expression cassette” is heterologous with respect to the host, i.e. particular nucleic acid sequence of this expression cassette does not occur naturally in the host cell and must be introduced into the host cell or into progenitor of the host cell by way of transformation. Expression of the nucleotide sequence can be regulated by a constitutive promoter or an inducible promoter that initiates transcription only when the host cell is open to a specific external stimulus. In the case of a multicellular organism, the promoter may also have specificity to a particular tissue, or organ, or developmental stage.

The term “heterologous” or “exogenous” nucleic acid means a nucleic acid never occurring in a wild-type host cell.

The term “endogenous” refers to a native protein or nucleic acid in its natural position within genome of the organism.

The term “specifically hybridizes”, as used herein, refers to an association between two single-stranded nucleic acid molecules or sufficiently complementary sequences such as to permit the hybridization under predetermined conditions commonly used in the art (sometimes the term “substantially complementary” is used).

“Transformation” is the process for introducing a heterologous nucleic acid into a host cell or organism. In particular, “transformation” means a stable integration of DNA moiety into genome of a target organism of interest.

The term or terms “transformed/transgenic/recombinant” refers to a host organism such as bacterium, plant, fungus, or animal, which was modified by introducing a heterologous nucleic acid moiety. This nucleic acid moiety may be either stably integrated into the host genome, or occur as an extrachromosomal moiety. Such an extrachromosomal moiety may be capable of self-replication. It should be understood that transgenic or stably transformed cells, tissues or organisms include both end products of the transformation process, but also transgenic progeny. Terms “non-transformed,” “non-transgenic,” “non-recombinant,” or “wild-type” refer to a natural host organism or host cell, for example, a bacterium or plant, that contain no heterologous nucleic acid moieties.

The term “autonomously luminous” or “autonomously bioluminescent” refers to transgenic organisms or host cells that are capable of bioluminescence without exogenous addition of luciferins, preluciferins, or precursors of preluciferins.

Nucleotides are designated according to their bases using the following standard abbreviations: adenine (A), cytosine (C), thymine (T) and guanine (G). Similarly, amino acids are designated by the following standard abbreviations: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile, I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

At a high level, the present disclosure is an advancement in the identification of one or more new variant(s) of luciferases and fungal luciferin biosynthesis enzymes, nucleic acids able to encode these enzymes, and proteins able to catalyze certain stages of the fungal luciferin biosynthesis. The disclosure also provides for application of nucleic acids for producing one or more enzymes in a cell or organism. Vectors comprising nucleic acid described in the present disclosure are also provided. In addition, the present disclosure provides expression cassettes comprising the nucleic acid as disclosed herein and regulatory elements necessary for nucleic acid expression in a selected host cell. Cells, stable cell lines, transgenic organisms (e.g. plants, animals, fungi, or microorganisms) including proteins, nucleic acids, vectors, or expression cassettes of the instant disclosure are also provided. The present disclosure also provides combinations of proteins and nucleic acids to obtain autonomously luminous cells, cell lines, or transgenic organisms. In one or more preferred embodiments, cells or transgenic organisms are capable to produce fungal luciferin from precursors. In some embodiments, cells or transgenic organisms are capable of autonomous bioluminescence. The present disclosure also provides a kit containing proteins, nucleic acids, vectors, or expression cassettes of the instant disclosure for producing luminous cells, cell lines, or transgenic organisms.

### Nucleic Acid Molecules

The present disclosure provides one or more nucleic acid molecules encoding luciferases and functional fragments thereof (for example, encoding truncated or extended luciferase variants). In some embodiments, the nucleic acid of the present invention encodes a luciferase at least 90% identical to SEQ ID NO: 1, or at least 90% identical to positions 40-267 of SEQ ID NO: 1, and comprises at least one amino acid substitution selected from T99P, T192S, A199P. In preferred embodiments, the nucleic acid of the present invention encodes a luciferase comprising the amino acid sequence of SEQ ID NO: 3. Nucleic acid molecules encoding homologs and mutants of said luciferases which are substantially similar to the above-mentioned luciferases also fall within the scope of the present disclosure. Each of these specific types of nucleic acid molecules of interest is disclosed in more detail in the experimental section below.

This instant disclosure also provides for nucleic acids encoding hispidin-3-hydroxylase and functional fragments thereof, mutants and homologs of these proteins, including shortened and elongated forms. In some embodiments, the nucleic acid of the present invention encodes a hispidin hydroxylase at least 90% identical to SEQ ID NO: 5 and comprises at least one amino acid substitution selected from D37E, V181I, A183P, S323M, M385K. In preferred embodiments, the nucleic acid of the present invention encodes a hispidin hydroxylase comprising the amino acid sequence of SEQ ID NO: 6. Nucleic acid molecules encoding homologs and mutants of the preferred hispidin-3-hydroxylases which are substantially similar to the above-mentioned hispidin-3-hydroxylase also fall within the scope of the present disclosure.

As used herein, a nucleic acid molecule is a DNA molecule, such as genomic DNA or cDNA, or an RNA molecule, such as an mRNA molecule. In some embodiments, the nucleic acid molecule of the present invention is a DNA molecule (or cDNA) having an open reading frame that encodes the luciferase of the present invention or hispidin-3-hydroxylase and is capable under suitable conditions (for example, under physiological intracellular conditions) of being expressed as a luciferase or hispidin-3-hydroxylase in a heterologous expression system.

In one or more embodiments, the nucleic acid molecule of the present disclosure is genetically engineered. Methods for obtaining nucleic acids are well known in the art. For example, the availability of amino acid sequence information (for example, SEQ ID NO: 3 or 6) or nucleotide sequence information (for example, SEQ ID NO: 10 or 13) makes it possible to prepare the isolated nucleic acid molecules of the present invention by oligonucleotide synthesis. Provided the information on amino acid sequence is available, several nucleic acids which are different from each other because of degeneracy of the genetic code can be synthesized (e.g., SEQ ID NO: 10 and 11, or SEQ ID NO: 13 and 14). Methods for selecting codon variants for the desired host are well known in the art.

Synthetic oligonucleotides may be prepared using the phosphoramidite method as would be understood. The obtained constructs can be purified by the methods well-known in the art, such as high-performance liquid chromatography (HPLC) or other methods, as described, for example, in Sambrook et al., Molecular Cloning: A Laboratory Manual, 2^{nd} Ed., (1989) Cold Spring Harbor Press, Cold Spring Harbor, N.Y., and according to instructions described in, for example, United States Dept. of HHS, National Institute of Health (NIH) Guidelines for Recombinant DNA Research. The long double-stranded DNA molecules of the present invention may be synthesized as follows: several smaller fragments with necessary complementarity, which contain suitable ends capable of cohesion with an adjacent fragment can be synthesized. Adjacent fragments may be bound by a DNA ligase in recombination-based methods, or during PCR.

The present disclosure also encompasses nucleic acids which are homologous, substantially similar, identical, or derived from nucleic acids encoding the proteins of the present invention. The nucleic acids of the present disclosure are in an environment that is different from an environment in which they exist under natural conditions; for example, they are isolated, their number is increased, they exist or are expressed in in vitro systems or in cells or organisms other than those in which they exist under natural conditions.

Alterations or differences in a nucleotide sequence between highly similar nucleotide sequences may represent nucleotide sequence substitutions that occur during normal replication or duplication. Other substitutions may be specifically calculated and inserted into a sequence for specific purposes, such as altering the codons of certain amino acids or the nucleotide sequence of the regulatory region. Such special substitutions can be made in vitro using various mutagenesis technologies or obtained in host organisms under specific breeding conditions which induce or select these changes. Such specially prepared variants of the sequence can be called “mutants” or “derivatives” of the original sequence.

The present disclosure also provides a nucleic acid encoding luciferase which is substantially similar to the amino acid sequence of which is shown in SEQ ID NO: 3. The nucleic acid encoding such a polypeptide or fragment thereof can be obtained by any of the plurality of known methods. The cDNA fragment of the present invention can be used as a hybridization probe against a cDNA library from the target organism under high stringency conditions. The probe can be a large fragment or a shorter degenerate primer. Nucleic acids having a similar sequence can be detected by hybridization under high stringency conditions, for example, at 50° C. or higher temperatures (for example, 60° C. or 65° C.), 50% formamide, 0.1×SSC (15 mM sodium chloride/1.5 mM sodium citrate), 0.1% SDS. Nucleic acids having regions substantially identical to the reference sequence, for example, allelic variants, genetically modified nucleic acid variants, etc. are bound to the reference sequence under highly stringent hybridization conditions. Such nucleotide sequences may be isolated using probes, in particular labelled probes complementary to the reference DNA sequence.

The present disclosure also provides a nucleic acid encoding hispidin-3-hydroxylase which is substantially similar to the amino acid sequence of which is shown in SEQ ID NO: 6. The nucleic acid encoding such a polypeptide or fragment thereof can be obtained by any of the plurality of known methods. The cDNA fragment of the present invention can be used as a hybridization probe against a cDNA library from the target organism under high stringency conditions. The probe can be a large fragment or a shorter degenerate primer. Nucleic acids having a similar sequence can be detected by hybridization under high stringency conditions, for example, at 50° C. or higher temperatures (for example, 60° C. or 65° C.), 50% formamide, 0.1×SSC (15 mM sodium chloride/1.5 mM sodium citrate), 0.1% SDS. Nucleic acids having regions substantially identical to the reference sequence, for example, allelic variants, genetically modified nucleic acid variants, etc. are bound to the reference sequence under highly stringent hybridization conditions. Such nucleotide sequences may be isolated using probes, in particular labelled probes complementary to the reference DNA sequence.

Mutant or derived nucleic acids may be obtained from a template nucleic acid selected from the above-described nucleic acids by modifying, deleting, or adding one or more nucleotides in the template sequence or a combination thereof to generate a variant of the template nucleic acid. Modifications, additions or deletions may be performed using any method known in the art (see for example Gustin et al., Biotechniques (1993) 14: 22; Barany, Gene (1985) 37: 111-123; and Colicelli et al., Mol. Gen. Genet. (1985) 199:537-539, Sambrook et al., Molecular Cloning: A Laboratory Manual, (1989), CSH Press, pp. 15.3-15.108), including error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, random mutagenesis, gene reassembly, gene site saturation mutagenesis (GSSM), synthetic ligation reassembly (SLR), or a combination thereof.

The modifications, additions and deletions may also be introduced by a method which includes recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deleted mutagenesis, restriction-selection mutagenesis, restriction-purification mutagenesis, artificial gene synthesis, ensemble mutagenesis, chimeric nucleic acid multimer creation, or a combination thereof.

Additionally, degenerate variants of nucleic acids which encode proteins of the present invention are provided. The degenerate nucleic acid variants include substitutions of nucleic acid codons with other codons which encode the same amino acids. In particular, degenerate variants of nucleic acids are generated to increase expression in a host cell. In this embodiment, nucleic acid codons that are not preferred or less preferred in host cell genes are substituted with codons that are abundantly present in the coding sequences of genes in the host cell where said substituted codons encode the same amino acid. Examples of degenerate variants of interest are described in more detail in the experimental section below.

Nucleic acids encoding truncated and extended variants of these luciferases also fall within the scope of the present disclosure. As used herein, these protein variants comprise amino acid sequences with the altered C-terminus, N-terminus, or both termini of a polypeptide chain.

Nucleic acids encoding truncated and extended variants of hispidin-3-hydroxylases also fall within the scope of the present invention. As used herein, these protein variants comprise amino acid sequences with the altered C-terminus, N-terminus, or both termini of a polypeptide chain.

In truncated variants, one or more (normally up to 27, more often 20 or less) amino acids may be removed from the sequence or substituted with any other amino acid. In particular, the sequence encoding a transmembrane domain from the N-terminus of luciferase can be completely or partially removed, modified, or replaced with an alternative transmembrane domain. The transmembrane domain can be identified using methods known in the prior art: for example, using algorithms described in [Krogh et al., Journal of Molecular Biology 2001, 305(3):567-580] and [Sonnhammer et al., Proceedings of the Sixth International Conference on Intelligent Systems for Molecular Biology, pages 175-182, Menlo Park, Calif., 1998. AAAI Press]. For analysis, software which is based on said algorithm and described in [http://www.cbs.dtu.dk/services/TMHMM/] can be used. In extended variants, the C- or N-terminus of a protein may contain additional amino acids. These additional amino acids may comprise purification tags (e.g., HisTag), detection tags (e.g., FLAG, HiBiT), solubility tags (e.g., S-tag), stabilization (e.g., SUMO), signal peptides, or functional protein domains (e.g., MBP or GST).

The above-mentioned modifications do not substantially change the spectral properties of luciferases or hispidin synthase, but can change intracellular localization, stimulate protein folding in host cells, decrease aggregation or modulate other biophysical protein properties, for example, thermostability or degradation half-life. In some embodiments, these modifications do not alter enzymatic or biochemical properties. All the above types of modifications and mutations are, as a rule, performed on the nucleic acid level.

The nucleic acid molecules of the present disclosure may encode the entire subject protein or a part thereof. Two- and single-stranded fragments can be obtained from a DNA sequence by chemical synthesis of oligonucleotides in accordance with standard methods, enzymatic restriction, PCR amplification, etc. In general, the size of DNA fragments will be at least approximately 15 nucleotides, normally at least approximately 18 nucleotides or approximately 25 nucleotides and may be at least approximately 50 nucleotides. In some embodiments, the subject nucleic acid molecules may have a size of approximately 100, approximately 200, approximately 300, approximately 400, approximately 500, approximately 600 or more. The subject nucleic acids may encode fragments of the subject proteins or complete proteins; for example, the subject nucleic acids may encode polypeptides of approximately 25 amino acids, approximately 50, approximately 75, approximately 100, approximately 125, approximately 150, approximately 200 amino acids, up to the complete length of the protein.

The subject nucleic acids may be isolated and obtained substantially free from other natural biological molecules and normally are “recombinant”, i.e. flanked by one or more nucleotides commonly not bound to a sequence in a chromosome existing in nature in the natural host thereof.

Also provided are nucleic acids which encode a fused protein comprising proteins of the present invention or fragments thereof which are described in detail below.

A vector and other nucleic acid constructs comprising the subject nucleic acids are also provided. Suitable vectors comprise viral and non-viral vectors, plasmids, cosmids, phages, etc., preferably plasmids, and are used for cloning, amplifying, expressing, transferring, etc. the nucleic acid sequence of the present invention to a suitable host. To prepare the constructs, the partial or full-length nucleic acid is inserted into a vector typically by means of DNA ligase attachment to a cleaved restriction enzyme site in the vector. Alternatively, the desired nucleotide sequence can be inserted by in vivo homologous recombination, typically by attaching homologous regions to the vector on the flanks of the desired nucleotide sequence. Regions of homology are added by ligation of oligonucleotides, or by polymerase chain reaction using primers comprising both the region of homology and a portion of the desired nucleotide sequence, for example. The vector, as a rule, has an origin of replication, which ensures replication thereof in the hosts as a result of being introduced into a cell as an extrachromosomal element. The vector may also contain regulatory elements providing the expression of the nucleic acid in the host and the generation of recombinant functional luciferase. The vector may also contain regulatory elements providing the expression of the nucleic acid in the host and the generation of recombinant hispidin-3-hydroxylase. In the expression vector, said nucleic acid is functionally linked to a regulatory sequences which may include promoters, enhancers, terminators, operators, repressors, silencers, insulators and inducers.

Expression cassettes or systems used inter alia for obtaining the subject luciferases or chimeric proteins based thereof, or for replication of the subject nucleic acid molecules are also provided. Expression cassettes or systems used inter alia for obtaining the subject hispidin-3-hydroxylases or chimeric proteins based thereof, or for replication of the subject nucleic acid molecules are also provided. The expression cassette may exist as an extrachromosomal element or may be integrated into the genome of the cell as a result of introduction of said expression cassette into the cell. For expression, the gene product encoded by the nucleic acid of the invention is expressed in any convenient expression system, including, for example, bacterial, yeast, plants, insects, amphibians or mammalian systems. In the expression cassette, the target nucleic acid is operably linked to regulatory sequences that can include promoters, enhancers, termination sequences, operators, repressors, and inducers. Methods for preparing expression cassettes or systems capable of expressing the desired product may be understood by a person skilled in the art.

Cell lines which sustainably express the proteins of the present invention can be selected by the methods known in the art (e.g. co-transfection with a selection marker, such as dhfr, gpt, neomycin or hygromycin allows the identification and isolation of the transfected cells that contain the gene integrated into a genome).

The above expression systems can be used in prokaryotic or eukaryotic hosts. Host cells, such as E. coli, B. subtilis, S. cerevisiae, insect cells or higher organism cells which are not human embryo cells, such as yeast and plants (for example, Arabidopsis thaliana, Nicotiana benthamiana, Physcomitrella patens, Nicotiana tabacum, Petunia hybrida, Populus canadensis), vertebrates, for example, COS 7 cells, HEK293, CHO, Xenopus oocytes, etc. may be used for production of the protein.

Nucleic acid molecules of the present invention may also be generated through advanced targeted mutation methods, akin to the way genetic alterations are achieved through precise guidance systems (e.g., CRISPR-Cas9). By designing specific sequences that correspond with key segments within a gene, specialized enzymes can be directed to initiate controlled breaks at predetermined sites. As the host cell activates its inherent repair processes, the resulting mending action introduces intentional mutations, insertions, or deletions within the gene's structure. This technique enables the capacity to craft customized variations in situ (i.e., within living cells) with exceptional precision and efficiency. By this methodology, nucleic acids of the present invention may be created through modification of related nucleic acids present within the host cell (i.e., encoding a protein having an amino acid sequence similar to the present invention).

When any of the above-referenced host cells, or other appropriate host cells or organisms are used to replicate and/or express the nucleic acids of the invention, the resulting replicated nucleic acid, expressed protein or polypeptide is within the scope of the disclosure as a product of the host cell or organism. The product may be recovered by any appropriate method known to a person having skill in the relevant art.

Also provided are small DNA fragments of the subject nucleic acids, that are useful as primers for PCR, rolling circle amplification, hybridization screening probes, etc. Larger DNA fragments are useful for production of the encoded polypeptide, as described previously. However, for use in geometric amplification reactions, such as PCR, a pair of small DNA fragments, i.e. primers, is used. The exact composition of the primer sequence is not critical to the scope of the instant disclosure (and thus not fully described herein), but for most applications the primers will hybridize to the subject sequence under stringent conditions, as known in the art. It is preferable to choose a pair of primers that will generate an amplification product of at least approximately 50 nucleotides, preferably at least approximately 100 nucleotides and extend to the entire nucleic acid sequence. Algorithms for the selection of primer sequences are generally known, and are available in commercial software packages. Amplification primers hybridize to complementary strands of DNA, and will prime towards each other.

The nucleic acid molecules of the present invention may also be used to identify expression of the gene in a biological sample. The manner in which one probes cells for the presence of particular nucleotide sequences, such as genomic DNA or RNA, is well-established in the literature. Briefly, DNA or mRNA is isolated from a cell sample. The mRNA may be amplified by RT-PCR, using reverse transcriptase to form a complementary DNA strand, followed by polymerase chain reaction amplification using primers specific for the subject DNA sequences. Alternatively, the mRNA sample is separated by gel electrophoresis, transferred to a suitable support, e.g. nitrocellulose, nylon, etc., and then probed with a fragment of the subject DNA as a probe. Other techniques, such as oligonucleotide ligation assays, in situ hybridization, and hybridization to DNA probes arrayed on a solid chip may also find use. Detection of mRNA hybridizing to the subject sequence is indicative of gene expression in the sample.

### Proteins

One or more aspects of the instant disclosure provides luciferases, as well as homologs, derivatives and mutants thereof, including full-length proteins and parts or fragments thereof. The preferred proteins are luciferases capable of catalyzing the oxidation of luciferin in the presence of oxygen. The oxidation reaction is independent of the presence of ATP, NAD(P)H and other metabolites in the medium. The subject proteins may differ from the known luciferases because the preferred proteins oxidize 3-hydroxyhispidin having the following structure:

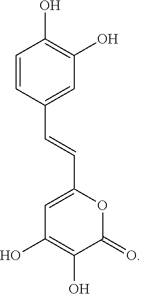 [SMILES: O=c1oc(/C=C/c2ccc(O)c(O)c2)cc(O)c1O]

Oxidation of luciferin by luciferase of the present invention is accompanied by the release of detectable light.

In some embodiments of the present disclosure, the light released during the reaction can be detected using conventional methods (for example, visual inspection, night vision, spectrophotometry, spectrofluorimetry, image photographic recording, special luminescence and fluorescence detection equipment, such as, for example, IVIS Spectrum In Vivo Imaging System (Perkin Elmer), etc.). The detected light may be emitted within the intensity range of 100 photon per second per cm² to easily visible light, for example, with an intensity of 1 cd, and bright light with an intensity of, for example, 100 cd or more.

The light emitted during oxidation of 3-hydroxyhispidin is preferably within the range of 400 to 700 nm, and predominately within the range of 450 to 650 nm, with an emission maximum at 520-590 nm. The subject proteins remain active at temperatures below 50° C., typically at temperatures up to 45° C., i.e. they remain active within the range of temperatures of 30-45° C.

The subject proteins have pH stability within the range of 4 to 10, and preferably within the range of 6.5 to 9.5. The optimum pH stability of the subject proteins falls within the range of 7.0 to 8.0, for example, 7.3 to 7.5.

The specific proteins of interest include one or more preferred luciferases having an amino acid sequence at least 90% identical to SEQ ID NO: 1 and at least one amino acid substitution selected from T99P, T192S, A199P. For example, but not considered a limitation, specific proteins of interest comprise the amino acid sequence shown in SEQ ID NO: 3, and described in more detail in the experimental section below.

Luciferases substantially similar to the luciferases described herein and functional fragments thereof are also provided. In many embodiments, the amino acid sequences of interest are significantly identical in the sequence, for example, at least 90% identical to SEQ ID NO: 3 (for example, 90%, 91%, 92%, 93%, 94%, 95%, 96%, 97%, 98%, 98% or 99% identical). In particular, this refers to the sequence of amino acids that provide protein functional regions, i.e. to the protein sequence located after the sequence of a transmembrane domain which is a part of natural luciferases of the present disclosure. The instant disclosure also provides mutants of the above referred proteins. Mutants may retain the biological properties of proteins from which they were obtained or may have biological properties that differ from those of wild-type proteins. The term “biological property” of proteins according to the present disclosure refers, without limitation, to ability to oxidize various luciferins; biochemical properties, such as in vivo and/or in vitro stability (for example, half-life); maturation rate; aggregation or oligomerization tendency as well as other similar properties. Mutations comprise alterations to one or more amino acids, deletions or insertions of one or more amino acids; N-terminus truncations or extensions, C-terminus truncations or extensions, etc. It is commonly understood, for example, that the functional effects of individual amino acid substitutions present in mutant proteins typically act cumulatively when the substitutions are combined into a single protein. Thus, the beneficial effects (i.e., increased luminescence) of the amino acid substitutions described in the present invention would be largely maintained when combined with other substitutions (see Examples 1 and 3). Mutants may be obtained using conventional molecular biology techniques, such as those described in detail above.

In the preferred embodiment, the target proteins are obtained using a synthesis method, for example, by expressing a recombinant nucleic acid encoding a sequence encoding the protein of interest in a suitable host, as described above. Any convenient protein purification procedures may be employed where suitable protein purification techniques are described in the appropriate reference guide (Guide to Protein Purification, (Deuthser ed.) (Academic Press, 1990). For example, a lysate may be prepared from the original source and purified using HPLC, exclusion chromatography, gel electrophoresis, affinity chromatography, etc.

The present invention also involves fusion proteins comprising the protein of the present disclosure or functional fragments thereof fused, for example, to a subcellular localization sequence (for example, nuclear localization signal, peroxisome localization signal, mitochondria, Golgi apparatus, etc.), signal peptide or any protein or polypeptide of interest. The fusion proteins may comprise, for example, luciferase of the present invention and a second polypeptide (“the fusion partner”) operably fused in-frame to the N-terminus and/or C-terminus of luciferase. Fusion partners include, but are not limited to, polypeptides that can bind to antibodies specific to the fusion partner (for example, epitope tags), antibodies or binding fragments thereof, polypeptides that provide a catalytic function or induce a cellular response, ligands or receptors or mimetics thereof, etc.

Hispidin-hydroxylases of this disclosure are proteins able to catalyze luciferin synthesis from preluciferin. The preferred proteins of interest include hispidin hydroxylases having an amino acid sequence at least 90% identical to SEQ ID NO: 5 and at least one amino acid substitution selected from D37E, V181I, A183P, S323M, M385K. For example, specific proteins of interest preferably comprise the amino acid sequence shown in SEQ ID NO: 6, and described in more detail in the experimental section below.

Hispidin-hydroxylase substantially similar to the above hispidin-hydroxylase and functional fragments thereof are also provided. In many embodiments, the amino acid sequences of interest are significantly identical to SEQ ID NO: 6, for example, at least 90% identical (for example, 90%, 91%, 92%, 93%, 94%, 95%, 96%, 97%, 98%, 98% or 99% identical). In some embodiments, the amino acid sequences have at least 90%, or at least 91%, or at least 92%, or at least 93%, or at least 94%, or at least 95%, or at least 96%, or at least 97%, or at least 98%, or at least 99% identical to SEQ ID NO: 6 for at least 350 amino acids.

The scope of the instant disclosure also covers fusion proteins, including proteins as disclosed herein. One or more embodiments of the protein of the instant disclosure could be operatively fused with intracellular localization signal (e.g. nuclear localization signal, localization signal in mitochondria, or in peroxisomes, or in lysosomes, or in Golgi apparatus, or in other cell organelles), signal peptide promoting protein isolation into intercellular space, transmembrane domain or with any protein or polypeptide (fusion partner) of interest. Fusion proteins may include operatively cross-linked, e.g. hispidin hydroxylase and/or hispidin synthase, and/or caffeylpyruvate hydrolase with fusion partner linked to C- or N-terminal. Non-limiting examples of fusion partners may include proteins of this invention having other enzymic function, antibodies or their linking fragments, ligands or receptors, luciferases able to use fungi luciferins as substrates in a bioluminescent reaction. In some embodiments a fusion partner and protein of the invention are physically cross-linked via linking sequence (peptide linker) promoting independent fusion protein folding and functioning.

In some embodiments fusion proteins include hispidin hydroxylase of the instant disclosure and luciferase able to oxidize fungal luciferin with luminescence emission, which are physically cross-linked via short peptide linker. Such fusion proteins can be used for obtaining bioluminescence in vitro and in vivo in the presence of a preluciferin (e.g. in the presence of hispidin).

### Transgenic Cells and Organisms

Transgenic cells and organisms comprising proteins of this disclosure, or expressing nucleic acids of this disclosure are also provided. For the purposes of this disclosure any suitable host cell may be used, including prokaryotic (e.g. Escherichia coli, Streptomyces sp., Bacillus subtilis, Lactobacillus acidophilus, etc.) or eukaryotic cells. In some embodiments, the host cell is not a human embryonic cell.

Nucleic acids may be introduced into a cell by transfection, transformation, gene gun delivery, microinjection, recombinant viral vector, or transconjugation. Techniques of nucleic acid (e.g. DNA) molecules transfer into cells are well-known and described in standard manuals, such as Sambrook et al. (Molecular Cloning: A Laboratory Manual, 3rd Ed., (2001) Cold Spring Harbor Press, Cold Spring Harbor, N.Y.).

DNA structures for homologous recombination include at least some portion of a nucleic acid of the instant disclosure, where the nucleic acid is operatively linked to homology regions for targeted locus. For random integration, homology regions are not necessary to facilitate recombination. Positive and negative selection markers may also be included. Methods for obtaining the cells comprising target gene modifications by homologous recombination are known in the art. Different techniques of mammal cells transfection are described, for example, in the paper Keown et al., Meth. Enzymol. (1990) 185:527-537).

Nucleic acids of the instant disclosure may also be introduced through in situ modification of related genes present within a host cell. For example, in situ modifications may be achieved using advanced targeted mutation methods (e.g., CRISPR-Cas9). Such techniques can provide precise modification of nucleic acids sequences in the host cell genome or extrachromosomal element.

Transgenic organisms of the present disclosure may be prokaryotic or eukaryotic organisms, including bacteria, cyanobacteria, fungi, plants and animals, where one or more nucleic acids of the instant disclosure are incorporated by human manipulation into one or more cells of the organism. Techniques for making transgenic organisms are generally known in the art. In some embodiments the transgenic organism may be a prokaryotic organism. Methods for transformation of prokaryotic host cells are well known in the art (see, for example, Sambrook et al. (Molecular Cloning: A Laboratory Manual, 2nd Ed., (1989) Cold Spring Harbor Laboratory Press and Ausubel et al., Current Protocols in Molecular Biology (1995) John Wiley & Sons, Inc). In other embodiments the transgenic organism could be a fungus, e.g. yeast. Yeasts are widely used as a host for heterologous gene expression (see, for example, Goodey et al., Yeast biotechnology, D R Berry et al., eds, (1987) Allen and Unwin, London, pp. 401-429, and Kong et al., Molecular and Cell Biology of Yeasts, E. F. Walton and G. T. Yarronton, eds, Blackie, Glasgow (1989) pp. 107-133). There are several yeast vectors available, including autonomously replicating plasmid vectors and integrating vectors, which require recombination with host genome for their maintenance.

In some embodiments the transgenic organism may be an animal. Embodiments of transgenic animals may be any animals which are not human, including mammals (e.g. mouse or rat), birds or amphibia. Other embodiments of transgenic animals may be obtained using transgenic techniques known in the art and described in standard manuals (such as: Pinkert, Transgenic Animal Technology: A Laboratory Handbook, 2nd edition (2003) San Diego: Academic Press; Gersenstein and Vinterstein, Manipulating the Mouse Embryo: A Laboratory Manual, 3rd ed, (2002) Nagy A. (Ed), Cold Spring Harbor Laboratory; Blau et al., Laboratory Animal Medicine, 2nd Ed., (2002) Fox J. G., Anderson L. C., Loew F. M., Quimby F. W. (Eds), American Medical Association, American Psychological Association; Gene Targeting: A Practical Approach by Alexandra L. Joyner (Ed.) Oxford University Press; 2nd edition (2000)). For example, transgenic animals may be obtained by homologous recombination to modify a specific endogenous locus, or alternatively by random integration into a genome. Vectors for stable integration include plasmids, retroviruses and other animal viruses, YAC, etc.

In other embodiments the transgenic organism may be a plant. Transgenic plants could be obtained, for example, by “gene gun”-based methods or by Agrobacterium-mediated transformation. Methods for obtaining transgenic plant cells are described in the U.S. Pat. Nos. 5,767,367, 5,750,870, 5,739,409, 5,689,049, 5,689,045, 5,674,731, 5,656,466, 5,633,155, 5,629,470, 5,595,896, 5,576,198, 5,538,879 and 5,484,956, the entirety of which are incorporated by reference herein in this disclosure. Methods for obtaining transgenic plants are summarized in the following reviews: Plant Biochemistry and Molecular Biology (eds. Lea and Leegood, John Wiley & Sons (1993) pp. 275-295 and Plant Biotechnology and Transgenic Plants (eds. Oksman-Caldentey and Barz) (2002) 719 p.

### Methods of Use

The polypeptides and nucleic acids of the present disclosure find use in a variety of different applications. For example, they are used as reagents for diagnostics, quality control, environmental tests and other similar assays in biotechnology and medicine. In addition, they find use in domestic and entertainment-oriented applications, for example, in the generation of bioluminescent transgenic cells, plants and animals which can be used as light sources is outlined herein.

For example, the nucleic acids of the instant disclosure may be used for detection of various external signals, for example, environmental conditions affecting plant health. For this embodiment, an expression cassette containing an environmentally-responsive element is introduced into a host cell or organism, where ambient conditions serve as a triggering event and modulated expression of bioluminescence provides a detectable indicator. The regulatory element may act by modulating gene transcription, RNA processing, post-translational modifications, or protein degradation. For example, by fusing luciferase to the signal of rapid protein degradation, the nucleic acids of the present disclosure may be used for indicating the presence of a toxic substance. Under this configuration, the presence of a toxic substance would cause a detectable reduction of bioluminescence.

The nucleic acid of the composition disclosed herein can be used to produce light-emitting transgenic cells, plants or animals. For example, to produce transgenic moss (Physcomitrella patens), nucleic acids of the present invention may be integrated into the genome under the control of a constitutive promoter (e.g., the promoter of AktI gene of rice), or an inducible promoter (e.g., the heat-sensitive promoter of Gmhsp17.3B gene of soybean). Genetically modified plants may serve as light sources when 3-hydroxyhispidin or related molecules are added to the medium or soil, or autonomously bioluminescent if 3-hydroxyhispidin biosynthesis occurs in the host cells. The preferred embodiment of luciferase(s) emit predominantly green light and thus are optimally suitable for the emission of light through photosynthetic plant tissues due to reduced absorption of such tissues in the green region of the visible spectrum.

The nucleic acids of the composition may also be used to visualize cell proteins, organelles, individual cells or tissues. For example, in order to visualize the migration of cancer cells in an organism, the nucleic sequences of luciferases are introduced into cancer cells as part of an expression cassette or expression vector. In other embodiments, nucleic acids are introduced into all organism cells, but are under the control of promoters active in cancer cells only. Since 3-hydroxyhispidin is capable of penetrating through cell membranes, the subject luciferases can be visualized in vivo in living organisms without tissue fixation and permeabilization. To visualize the development of cancer tumors and metastasis, imaging equipment suitable for detecting bioluminescence may be used.

In some embodiments, hispidin hydroxylases are applied in vitro and in vivo to obtain luciferin, which is oxidized by bioluminescent fungi luciferases, their homologs and mutants with luminescence emission. Therefore, one or more embodiments of the present disclosure provided for application of hispidin hydroxylases of this disclosure to catalyze the transformation of hispidin (preluciferin) having the structural formula:

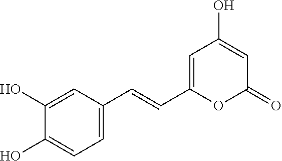 [SMILES: O=c1cc(O)cc(/C=C/c2ccc(O)c(O)c2)o1]

into 3-hydroxyhispidin (luciferin) having the structural formula:

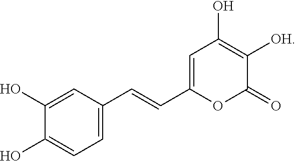 [SMILES: O=c1oc(/C=C/c2ccc(O)c(O)c2)cc(O)c1O]

Method for obtaining fungal luciferin from preluciferin includes combination of at least one molecule of hispidin hydroxylase with at least one molecule of 6-(2-arylvinyl)-4-hydroxy-2H-pyran-2-one. The reaction is carried out in physiological conditions in vitro and in vivo at the temperature from 20 to 42° C. The reaction could be carried out in cells, tissues and host organisms expressing hispidin hydroxylase. In preferred embodiments the said cells, tissues and organisms comprise sufficient amounts of NAD(P)H and molecular oxygen to carry out the reaction. Exogenously delivered 6-(2-arylvinyl)-4-hydroxy-2H-pyran-2-one or endogenous 6-(2-arylvinyl)-4-hydroxy-2H-pyran-2-one produced in cells, tissues and organisms could be used in the reaction. The 6-(2-arylvinyl)-3,4-dihydroxy-2H-pyran-2-one obtained by the reaction may be used to produce bioluminescence in in vitro and in vivo systems comprising a functional luciferase.

In the presence of hispidin hydroxylase and luciferase, the occurrence of detectable luminescence is indicative of hispidin or its functional analogue. In some embodiments a combination of the nucleic acids encoding hispidin hydroxylase luciferase are used. The combination is widely applicable for labeling organisms, tissues, cells, cell organelles or proteins by bioluminescence. In some embodiments instead of a combination of hispidin hydroxylase and luciferase, alternatively a fusion protein of hispidin hydroxylase and luciferase may be used.

The combinations are also applicable for producing transgenic luminous organisms. Autonomously bioluminescent transgenic organisms are of special interest. Higher and lower plants, including flowering plants and mosses are non-limiting examples. In some embodiments to obtain autonomously luminescence-producing organisms, nucleic acids able to express hispidin synthase are also introduced into the organism. In some embodiments instead of combination of nucleic acids coding hispidin hydroxylase and luciferase the nucleic acid coding fusion protein of these two enzymes is used.

All publications, patents and patent applications mentioned in this specification, including but not limited to PCT/RU2016/000229 filed 25 Feb. 2015, entitled: METHOD AND REAGENTS FOR DETECTING LUCIFERASE ACTIVITY, PCT/RU2017/050125 filed 30 Jan. 2017, entitled: NOVEL LUCIFERASES AND METHODS FOR USING SAME, PCT/RU2019/050097 filed 27 Jun. 2019, entitled: ENZYMES OF LUCIFERIN BIOSYNTHESIS AND USE THEREOF, U.S. application Ser. No. 17/135,163 filed 28 Dec. 2020, entitled: ENZYMES OF LUCIFERIN BIOSYNTHESIS AND USE THEREOF, and U.S. application Ser. No. 18/244,763 filed 11 Sep. 2023, entitled: ENZYMES OF LUCIFERIN BIOSYNTHESIS AND USE THEREOF are incorporated herein by reference herein in their respective entireties. Though one or more embodiments of the innovation as described herein have or has been described in considerable details by illustration and example for purposes of clarity, it is obvious to those skilled in the art, based on the ideas disclosed in this invention, that some alterations and modifications could be introduced without departing from the spirit and scope of the proposed embodiments of the development.

### EXAMPLES

### Example 1. Discovery of Beneficial Amino Acid Substitutions in Luciferase

A library of amino acid substitutions was created applying mutagenesis to a gene encoding the luciferase derived from Neonothopanus nambi (SEQ ID NO: 8). pF4Ag shuttle vector (Promega) was used for gene expression in E. coli (T7 promoter-driven expression) and mammalian cells (CMV promoter-driven expression). The pF4Ag vector backbone introduces a C-terminal 3×-FLAG-HiBiT tag. Site-directed mutagenesis was used to substitute consensus amino acids at selected position in the luciferase sequence. Gene expression was performed in the E. coli strain KRX (Promega), which enables rhamnose-inducible expression of T7 RNA polymerase.

Colonies (in triplicate) were used to inoculate LB broth containing ampicillin (100 g/ml) and cultures grown overnight in 96-well microtiter plates (20 hours, 37° C.). Cultures were diluted into fresh LB containing 0.1% rhamnose and ampicillin and grown for 3 hours at 37° C. 50 μl of the induced culture was lysed for 5 minutes at ambient temperature with a reagent consisting of 25 mM HEPES pH 7.5 and 0.3×PLB (E1941 Promega). 50 μl of lysate was combined with 50 μl luciferin (100 μM) in the lysis buffer and samples were measured for total luminescence using a GloMax®-Multi+luminometer. (Promega).

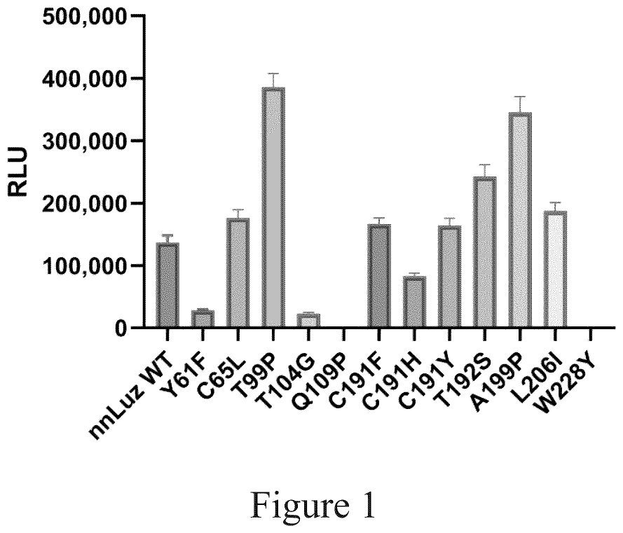

Substitutions at 12 positions were examined in E. coli lysates by this method. Of these, only three showed significant improvements in the production of bioluminescence (T99P, T192S, A199P) (FIG. 1). The three substitutions were combined into a single amino acid sequence to yield nnLuz_v3 (SEQ ID NO: 2).

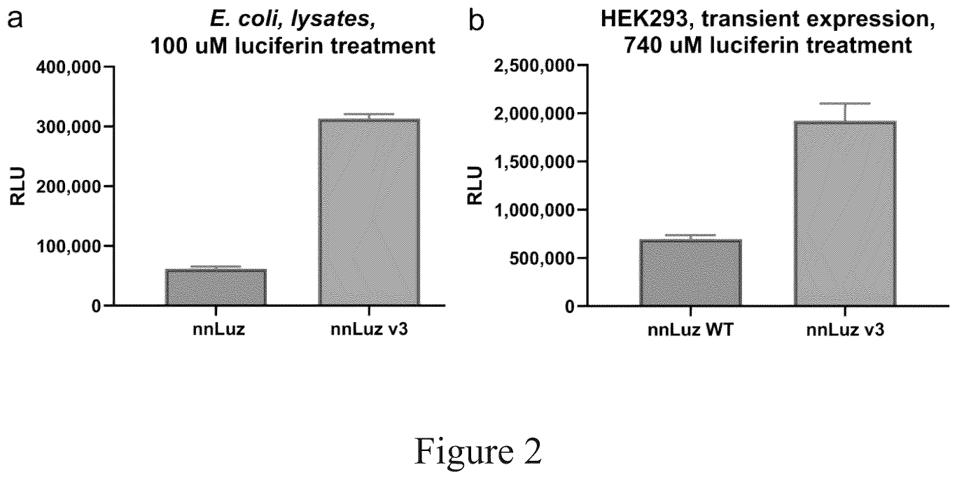

Production of luminescence by nnLuz_v3 was measure in E. coli lysates by the above method, and in mammalian cells (HEK293 cells). The HEK293 cells were transfected using the FugeneHD transfection reagent (Promega) according to the manufacturer's instructions. Cells were grown overnight under CO2 at 37° C. in 96-well white assay plates. For luminescence measurements, media was removed and replaced with 100 μl of CO2 independent media (Life Technologies 18045) containing 10% FBS. 33 μl of luciferin was added to cells (740 μM final concentration). The luminescence was measured using a GloMax-Multi+luminometer (Promega). Production of bioluminescence by nnLuz_v3 was substantially greater than the parental nnLuz (nnLuz_WT; SEQ ID NO: 1) in both E. coli (measured from lysates) and in mammalian cells (measured from living cells) (FIG. 2).

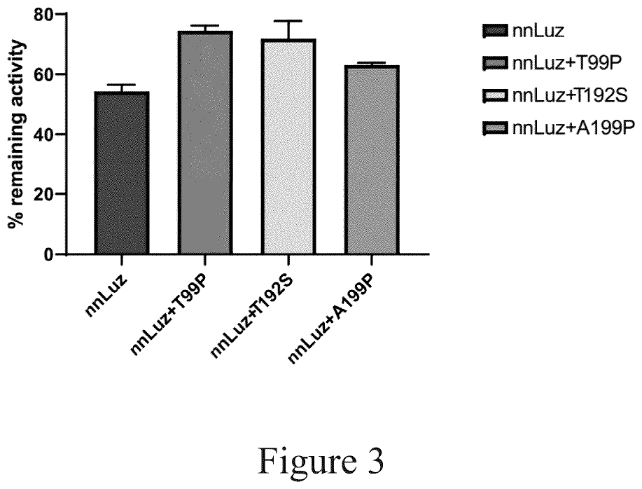

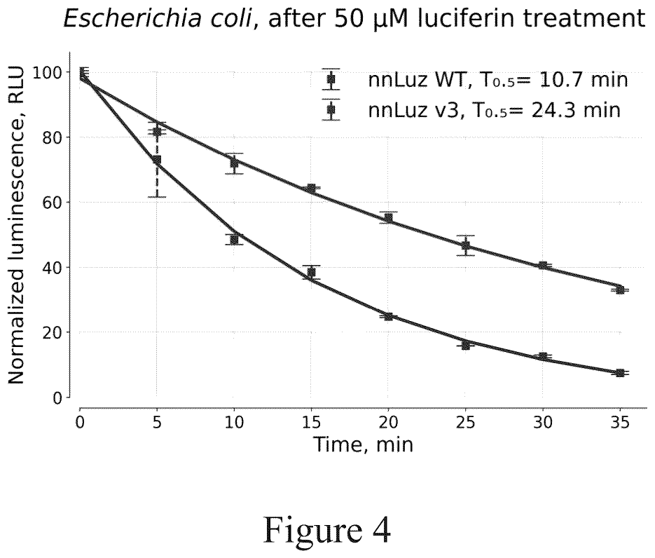

To assess the effect on enzyme thermostability by the amino acid substitutions, lysates of transformed E. coli were prepared by sonication and cleared by centrifugation. The cleared lysates were diluted 1:100 into Tris-buffered saline with 0.01% BSA and 0.1% n-dodecyl β-D-maltoside. 100 μl of each diluted lysate were added to a 96-well PCR plate. Samples were incubated at 22° C. and at various time points aliquots were placed on ice. When all of the samples were processed, 50 μl of each was combined with 50 μl of 100 μM luciferin in Tris-buffered saline with 0.01% BSA and 0.1% n-dodecyl β-D-maltoside. Samples were immediately placed in a GloMax®-Multi+luminometer (Promega) to measure bioluminescence. This method revealed that the luciferases having amino acid substitutions T99P, T192S, or A199P individually were more stable than the unmodified parental luciferase (FIG. 3). The combination of all three substitutions in nnLuz_v3 also produced greater enzyme stability (FIG. 4).

### Example 2. Discovery of Additional Beneficial Amino Acid Substitutions in Luciferase

Random mutagenesis of a gene encoding nnLuz_v3 (SEQ ID NO: 9) was achieved using Diversify PCR Random Mutagenesis Kit (630703 Takara) according to manufacturer's instructions. A library containing an average of 2.5 nucleotide changes per gene was used to transform KRX E. coli. 5,000 colonies were picked into 96-well plates and grown at 37° C. After overnight growth, cultures were diluted 20× into LB broth containing 0.1% Rhamnose and grown for 4 h at 37° C.

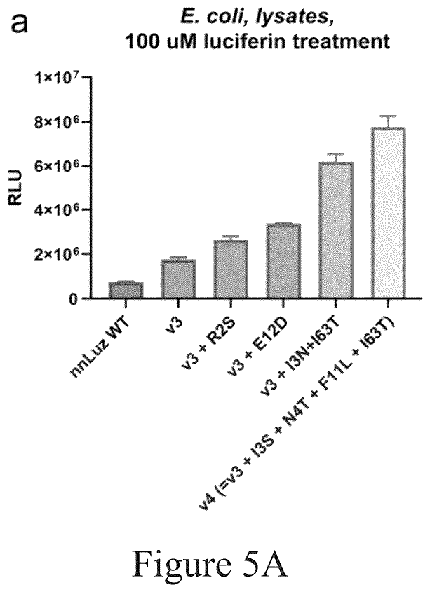

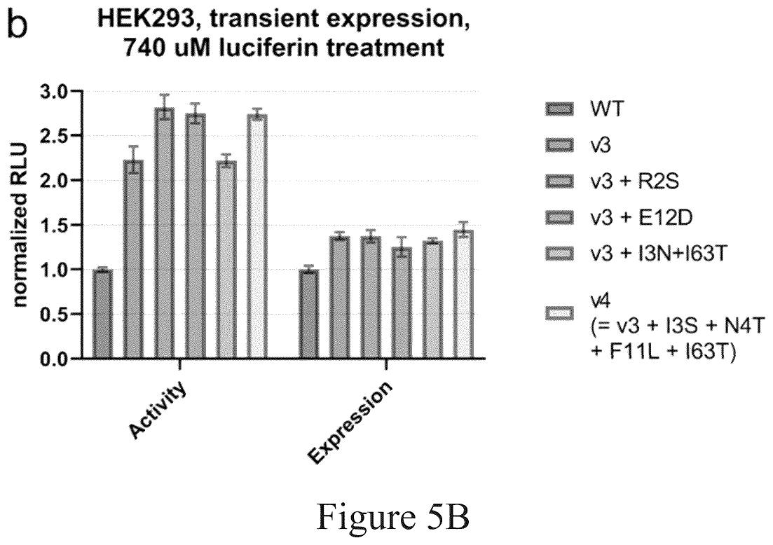

The sample plates were assayed for activity using a robotic screening platform as follows: 50 μl of induced cells were combined with 50 μl of PLB lysis buffer (0.3×PLB+25 mM HEPES pH 7.5). The plates were incubated for 5 min, followed by addition of 100 μl assay reagent containing 200 μM luciferin in PLB buffer. The plates were then transferred to a ClarioStar Plate reader (BMG) and luminescence measured. Clones that produced greater than 1.5-fold higher activity relative to nnLuz_v3 were assayed in a secondary screen following the same procedure. Selected amino acid substitutions identified in the mutagenesis library were further evaluated for production of luminescence in E. coli and mammalian cells using the procedures described in Example 1. Several substitutions were found to further increase bioluminescence above the level of nnLuz_v3 (FIGS. 5A-5B). The three substitutions of nnLuz_v3 were combined with I3S, N4T, F11L, and I63T into a single amino acid sequence to yield nnLuz_v4 (SEQ ID NO: 3).

Luminescence generated by the HiBiT tag was measured to estimate relative protein expression in HEK293 cells. 100 μl of HiBiT lytic reagent (Promega N3030) was added to 100 μl of HEK293 cells followed by 10-minute incubation on an orbital shaker at ambient temperature. Luminescence was subsequently read using a Glomax Multi+luminometer (Promega). This method indicated a lower variation in protein expression than evident for luciferase activity (FIGS. 5A-5B), suggesting that the amino acid substitutions had a greater influence on enzyme activity.

### Example 3. Discovery of Beneficial Amino Acid Substitutions in Hispidin Hydroxylase

A library of amino acid substitutions was created applying mutagenesis to a gene encoding the hispidin hydroxylase derived from Neonothopanus nambi (SEQ ID NO: 12). This was achieved by the same method as described in Example 1. Activity was evaluated in HEK293 cells co-transfected with a gene encoding nnLuz_v3. Bioluminescence was measured as described in Example 1, except with addition of hispidin instead of luciferin.

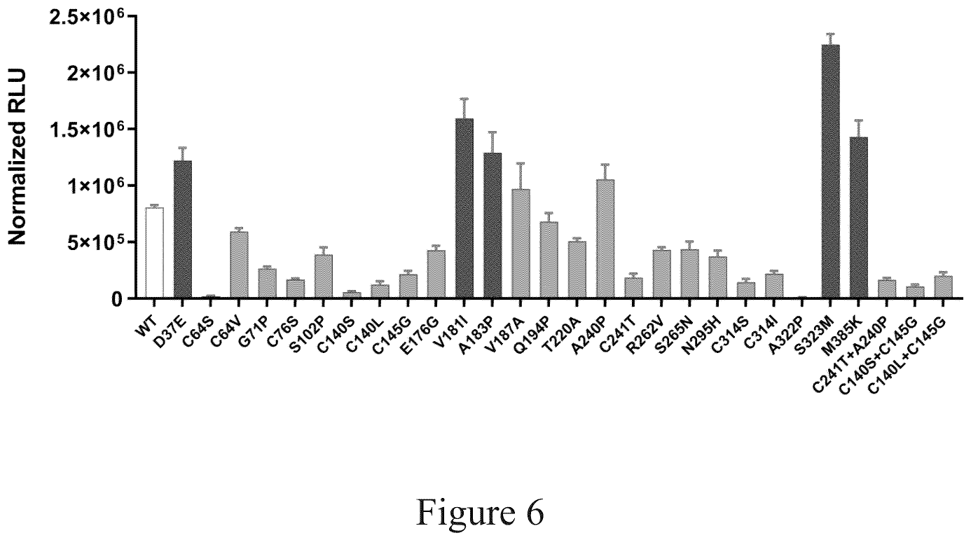

To estimate specific activity, both luciferase activity and HiBiT were measure for each sample (see Example 2). Activity of each variant was then normalized to the expression level (i.e., luciferase bioluminescence/HiBiT signal). Substitutions at 28 positions were examined by this method. Of these, only five (D37E, V181I, A183P, S323M, M385K) showed significantly increased bioluminescence over the parental enzyme (nnH3H_WT; SEQ ID NO: 5) (FIG. 6).

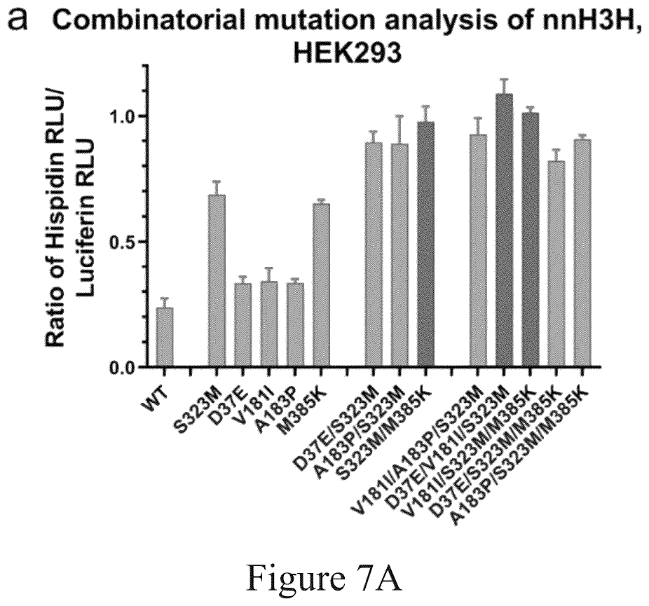

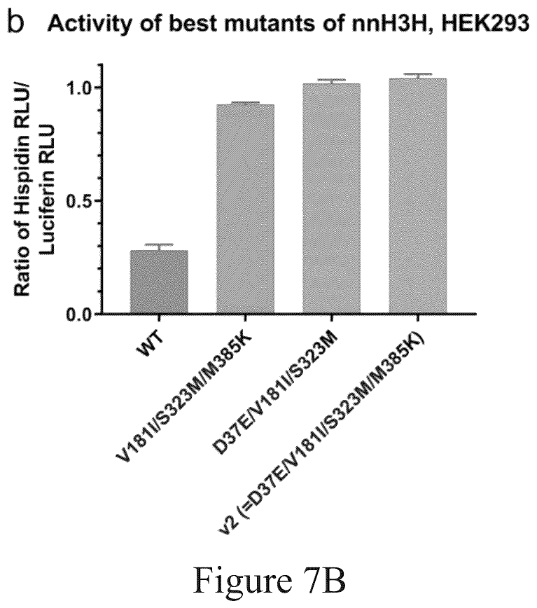

Various combinations of these substitutions were used to create composite amino acid sequences. Evaluation of these in HEK293 cells revealed that the substitutions acted in a cumulative fashion, i.e., the combinations were more effective than the individual substitutions (FIGS. 7A-7B). The most effective configurations were derived from the combination of three or four substitutions (FIGS. 7A-7B). The four substitutions D37E, V181I, S323M, and M385K were combined into a single amino acid sequence to yield nnH3H_v2 (SEQ ID NO: 6).

### Example 4. Construction of Plasmids for Optimized Expression in Yeast, Mammalian Cells, and Plants

Coding sequences of nnLuz_v4 (SEQ ID NO: 10) and nnH3H_v2 (SEQ ID NO: 13) were optimized for the expression in N. benthamiana, P. pastoris, and Homo sapiens (SEQ ID NO: 11 and 14, respectively). Synthetic DNA fragments were flanked by BpiI restriction sites designed to leave AATG-GCTT overhangs, compatible with the modular Golden Gate-based assembly standard. Golden Gate assembly was performed in the T4 ligase buffer (Thermo Fisher) containing 10 U of T4 ligase, 20 U of either BsaI or BpiI (Thermo Fisher) and ˜100 ng of each DNA part. Typically, Golden Gate reactions were performed according to “troubleshooting” cycling conditions described: 25 cycles (90 sec at 37° C., 180 sec at 16° C.), then 5 min at 50° C. and 10 min at 80° C.

Correct DNA assembly was typically confirmed by Sanger sequencing, and in some cases additionally by Nanopore or Illumina-based whole plasmid sequencing. DNA assembly and whole-plasmid sequencing was typically obtained from Cloning Facility. Plasmids for expression in yeast were based on Golden Gate-compatible backbones with GAP promoter and AOX terminator driving expression of the inserted gene, and different selectable markers (kanamycin resistance, hygromycin resistance, zeocin resistance, except for the NpgA expression plasmid which had HIS4-based selection cassette and homology arms for OLE1 locus.

Plasmids for expression in mammalian cells had the following insert structure: pCMV—gene—tSV40, and were assembled using DVK_AF vector as the backbone (CIDAR MoCLo Parts Kit #1000000059). In plasmids for expression in plants, nnLuz variants were cloned into MoClo Level 1-like vector under the control of the 1.3 kb constitutive 35S promoter from cauliflower mosaic virus with 5′ UTR of TMV omega virus and ocs terminator from Agrobacterium tumefaciens. HispS variants and nnCPH were cloned into Level 1-like vector under the control of the 0.4 kb constitutive 35S promoter from cauliflower mosaic virus with 5′ UTR of TMV omega and ocs terminator from A. tumefaciens. nnH3H variants were cloned into Level 1-like vector, under the control of the constitutive FMV promoter from figwort mosaic virus and nopaline synthase terminator from A. tumefaciens. NpgA gene was cloned into a Level 1-like vector, under the control of the constitutive CmYLCV 9.11 promoter from Cestrum yellow leaf curling virus and ATPase terminator from Solanum lycopersicum.

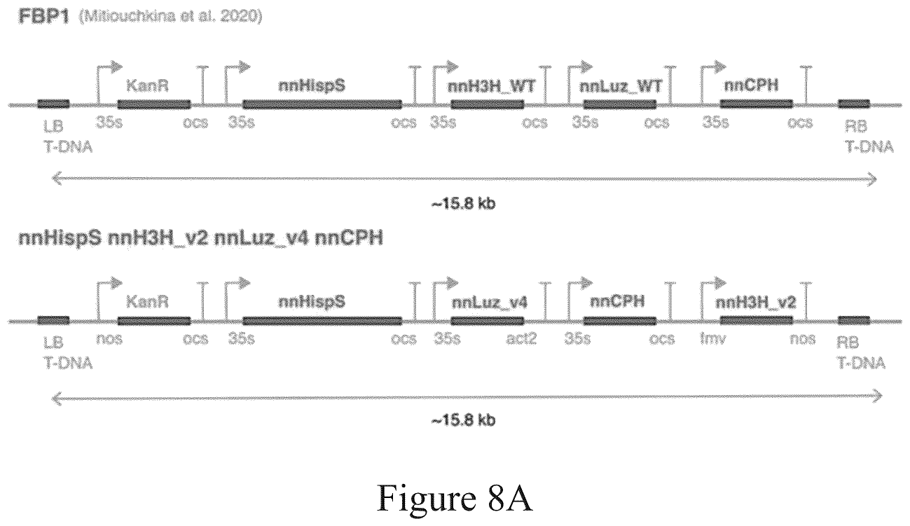

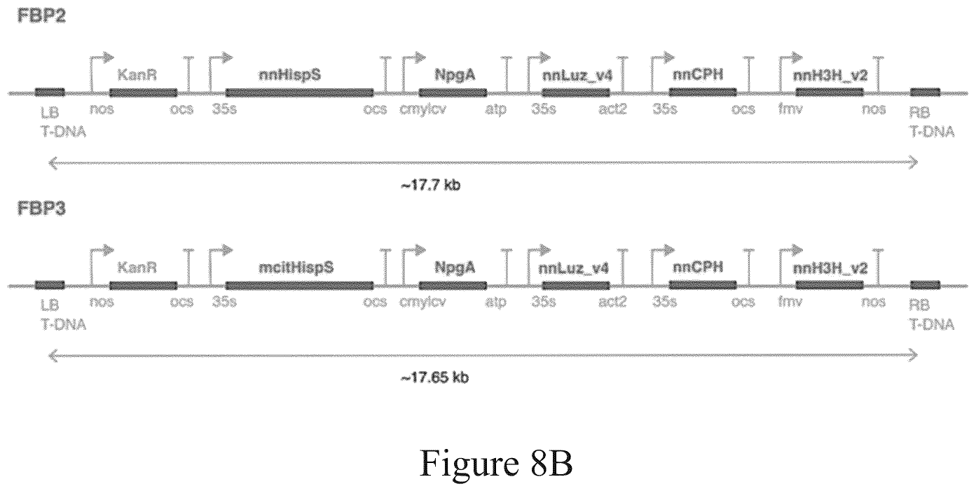

These Level 1 plasmids were then digested by BpiI and assembled together into several Level M-like backbones, which then were digested by Bsa and assembled together into Level P-like vectors resulting in polycistronic cassette of the following order: xxHispS-NpgA-nnLuz WT/v4-nnCPH-nnH3H WT/v2. This gene cluster was preceded by a kanamycin resistance cassette for selection in plants. The entire construct, consisting of the kanamycin cassette plus luminescence genes, was flanked by A. tumefaciens insertion sequences to facilitate Agrobacterium-mediated random integration of the construct into plant genomes (FIGS. 8A-8B). The construction method resulted in addition of two additional amino acids (Gly-Ser) onto the C-terminus of both nnLuz_v4 (SEQ ID NO: 4) and nnH3H_v2 (SEQ ID NO: 7). Plasmid encoding FBP1 (pX037; Addgene plasmid #167156) was obtained as described in [1]. To assess function within bioluminescent pathways, the modified enzymes were expressed with gene sets encompassing pathway versions, for example, FBP2 (nnHispS, nnH3H_v2, nnLuz_v4, nnCPH, NpgA), and FBP3 (mcitHispS, nnH3H_v2, nnLuz_v4, nnCPH, NpgA).

### Example 5. Enhanced Expression of Bioluminescence in Yeast

Integration of plasmids into the genome of Pichia pastoris GS115 was targeted at the GAP promoter locus. Linearized plasmids were used for the transformation of electrically competent yeast cells. Colonies were selected using 200 mg/ml of G418 or 200 mg/ml of hygromycin or 50 mg/ml of zeocin. PCR-based screening of yeast colonies was done by heating up colonies in 10 μl of 20 mM NaOH at 90° C. for 7 min and then using 1 μl of the resulting solution for direct PCR. For luminescence imaging, yeast culture grown on plate was resuspended in 50 μL of 1M sorbitol. 5 μL of the suspension was then added in 3 replicates to YPD agar plates lacking antibiotics. Plates were incubated at 30° C. for 20-24 h, and then 5 μL of 100 mM caffeic acid solution in the DPBS buffer, or 100 μM of hispidin solution in DPBS, or 100 μM of luciferin solution was added to each yeast strain. Imaging was performed in Fusion Pulse (Vilber, France), with exposure of 0.1 or 5 sec every 3 or 5 min for 1.0-1.5 hours—for caffeic acid treatment, with exposure of 20 sec for 20 mins—for hispidin treatment, with exposure of 0.5 sec every 10 sec for 10 mins in case of luciferin treatment.

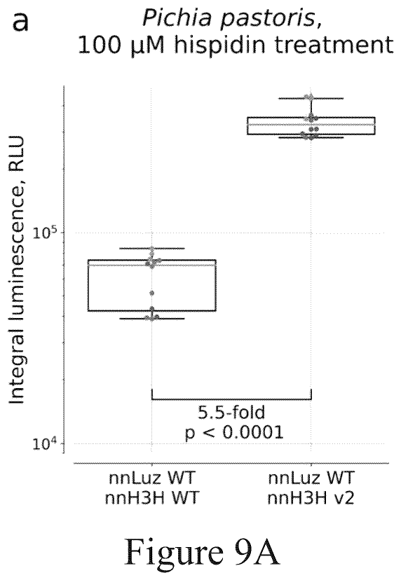

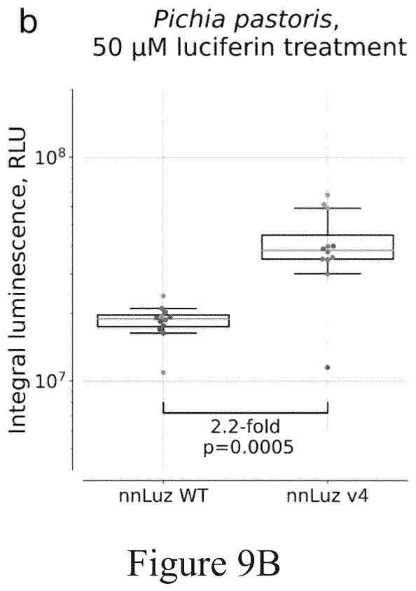

Processing of images was performed using FiJi ImageJ distribution (version 1.53t) 20 and custom Python scripts (Python version 3.8). For luminescence quantification mean values in the region of interest after background subtraction were used. Background subtraction was performed using the following formula: signal=signalraw−(backgroundmean−3*backgroundSD). Upon addition of 50 μM luciferin, cells expressing nnLuz_v4 produced greater bioluminescence relative to cell expressing nnLuz_WT (FIGS. 9A-9B). Similarly, upon addition of 100 μM hispidin, cells expressing both nnH3H_v2 and nnLuz_WT produced greater bioluminescence relative to cells expressing nnH3H_WT and nnLuz_WT.

To evaluate stability of nnLuz_WT and nnLuz_v4 within yeast cells, strains expressing nnLuz WT or nnLux_v4 were grown on plates for 24 hours, then resuspended in PBS to final OD (600 nm)=5 and 50 μl was placed in wells of a 96-well PCR plate. Different portions of the plate were incubated at different temperatures for 10 minutes using a gradient thermal cycler. The plate were then placed on ice for 5 minutes and 10 μl of 500 μM luciferin (2% DMSO, 10 mM thioglycolic acid, 0.1% DDM in DPBS) were added to each well. Then 25 μl of suspension from each well was transferred to a black 96-well plate containing 0.2 M sodium phosphate buffer with 0.5 M Na2SO4,pH=8.

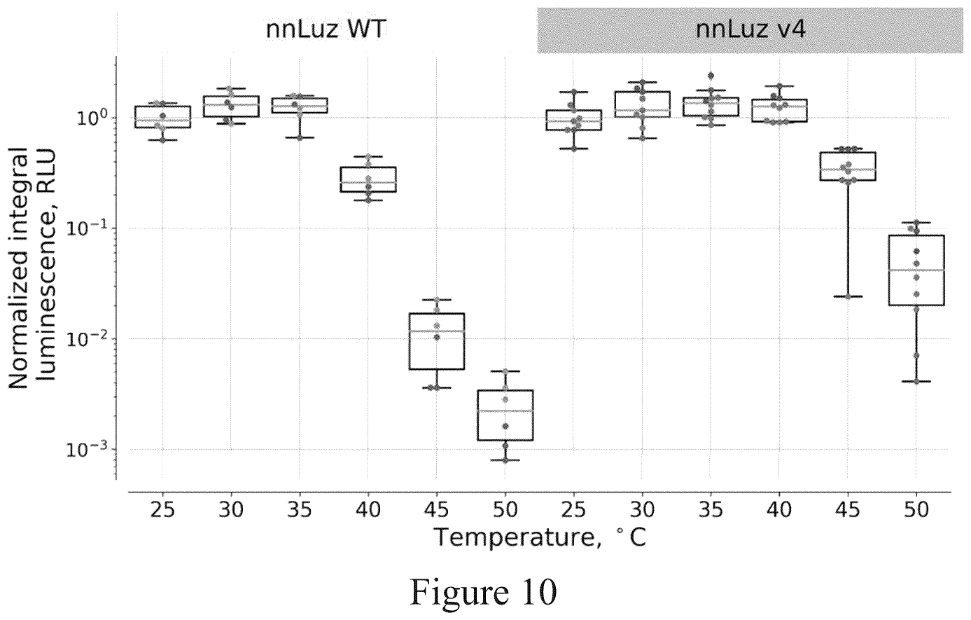

With incubation at 40° C., the luminescence by nnLuz_WT was reduced by >3-fold, whereas no loss of activity was evident for nnLuz_v4. Incubation at 45° C. resulted in 100-fold reduction of luminescence by nnLuz_WT, whereas the reduction was only 3-fold by nnLuz_v4 (FIG. 10). Consistent with Example 1 showing greater stability in cell lysates, these results demonstrate that the modified luciferase is also more stable within cells.

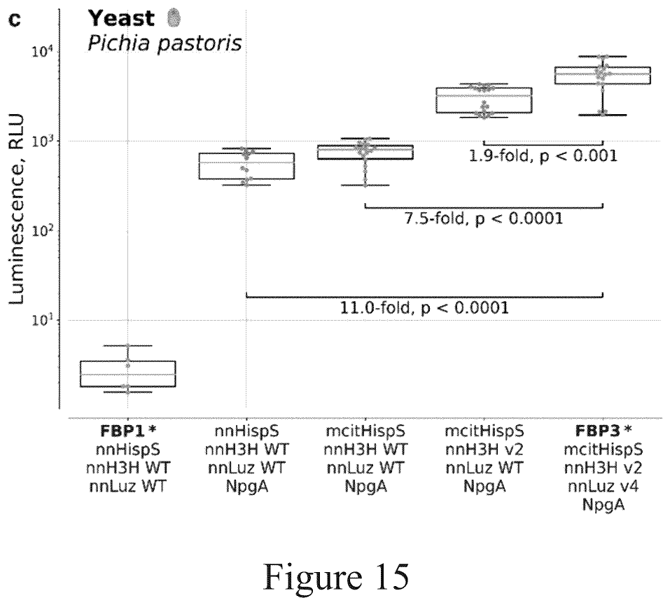

To evaluate the function of the modified enzymes within a bioluminescent pathway, nnH3H_v2 and nnLuz_v4 were expressed with genes for mcitHispS and NpgA (FIG. 15). When combined with nnLuz_WT, nnH3H_V2 produced 3.9-fold greater luminescence relative to nnH3H_WT. nnLuz_v4 produced 1.9-fold greater luminescence relative to nnLuz_WT when combined with nnH3H_v2.

### Example 6. Enhanced Expression of Bioluminescence in Plants

For introducing bioluminescent genes into plant cells, plasmids were transformed into competent cells of Agrobacterium tumefaciens AGL0 and clones were selected on LB agar plates containing 50 mg/L of rifampicin and an additional antibiotic, depending on the plasmid used for transformation (200 mg/L of carbenicillin, 50 mg/ml of kanamycin, or 100 mg/ml spectinomycin). Individual colonies were then inoculated into 10 mL of LB medium containing the same concentration of antibiotics. After overnight incubation at 28° C. with shaking at 220 rpm, cultures were centrifuged at 2900 g, resuspended in 25% glycerol and stored as glycerol stocks at −80° C.

To evaluate production of bioluminescence in plant cells, BY-2 cells derived from Nicotiana tabacum were grown in BY-2 medium (MS with 0.2 mg/L 2.4D, 200 mg/L KH2PO4, 1 mg/L thiamine, 100 mg/L myo-inositol, and 30 g/L sucrose) at 27° C. by shaking at 130 rpm in darkness. Each week, 2 ml of culture were transferred into new 200 ml of BY-2 medium.

To transform the BY-2 cells, one-week-old culture was pelleted in black 96-well plates to create cell packs and infiltrated by a mixture of several agrobacterial strains containing binary vectors. One of the strains encoded silencing inhibitor P19 (OD600=0.2), and others encoded bioluminescence genes (OD600=0.5). Plates were incubated at 80% humidity at 22° C. for 72 h before measurements of luminescence. Comparison of different sets of enzymes was done by co-infiltrating BY-2 cells with agrobacteria individually encoding bioluminescence enzymes.

Transient gene expression was achieved Nicotiana benthamiana leaves by agroinfiltration. On the day before agroinfiltration, glycerol stocks of agrobacteria were inoculated into 10 mL of LB containing 100 μM of acetosyringone, 50 mg/L of rifampicin and an additional antibiotic, depending on the plasmid which were used. The cultures were grown in the dark overnight at 28° C. with shaking at 220 rpm. The cultures were then centrifuged at 2900 g, resuspended in MMA buffer (10 mM MES, 10 mM MgCl2, 200 μM acetosyringone), and incubated at 28° C., 100 rpm for 3-4 h. Next, optical density at 600 nm was measured and used to dilute each culture to the optical density of 0.6. In addition, suspension of agrobacterium containing a plasmid encoding pNOS-P19-tOCS was added at the optical density of 0.2. Authors then mixed agrobacterial strains to infiltrate leaves of 4-6-week-old N. benthamiana, using a 1 mL medical syringe without needle. At least three different plants were infiltrated for each.

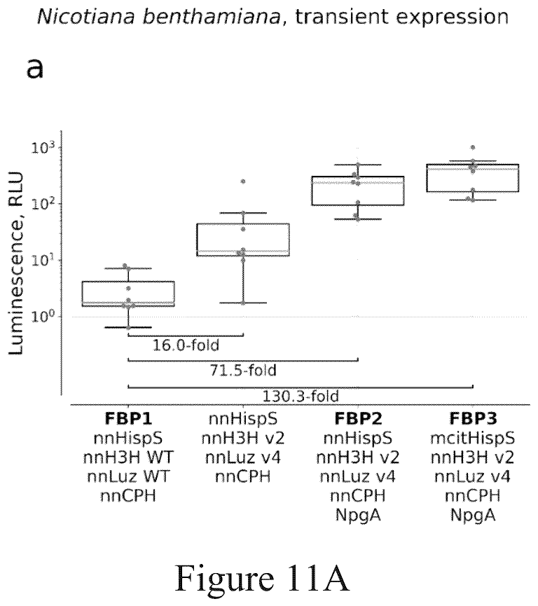

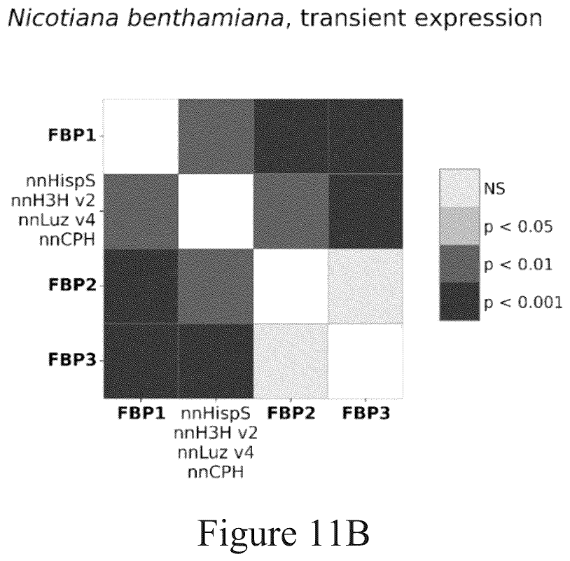

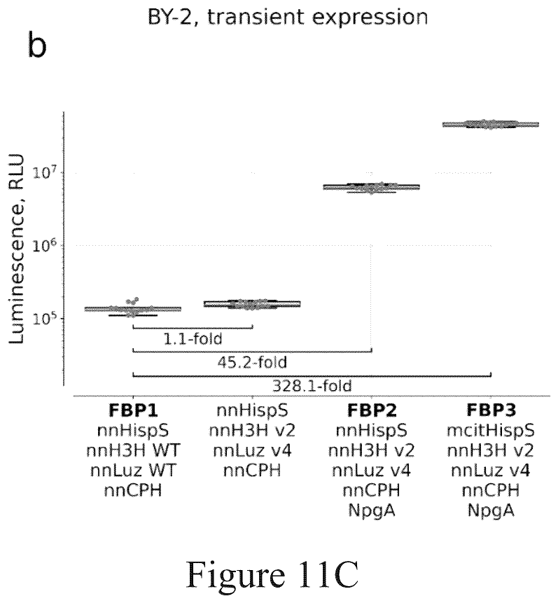

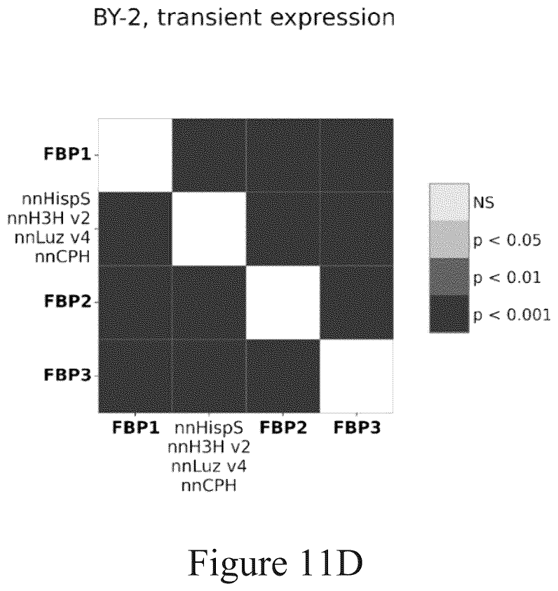

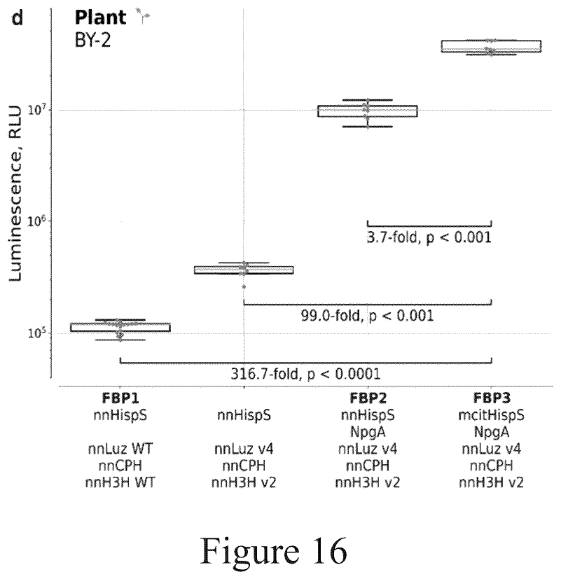

These methods were employed to assess the production of bioluminescence by transient gene expression of nnH3H_v2/nnLuz_v4 relative to their unmodified counterparts (FIGS. 11A, 11B, 11C, 11D). In BY-2 cells, the modified enzymes produced a small but significant increase in bioluminescence. In another experiment, the modified enzymes produced a 3.2-fold increase in bioluminescence (FIG. 16). In contrast, the modified enzymes produced a 16-fold increase when transiently expressed in leaves.

To achieve stable genetic transformation in a plant (N. benthamiana), A. tumefaciens strains containing the selected bioluminescent genes were grown in flasks on a shaker overnight at 28° C. in LB medium supplemented with 25 mg/L rifampicin and 50 mg/L kanamycin. Bacterial cultures were diluted in liquid Murashige and Skoog (MS) medium to an optical density of 0.6 at 600 nm. Leaf explants used for transformation experiments were cut from 2-week-old tobacco plants and incubated with bacterial culture for 20 min. Leaf explants were then placed onto filter paper overlaid on MS medium (MS salts, MS vitamin, 30 g/L sucrose, 8 g/L agar, pH 5.8) supplemented with 1 mg/L 6-benzylaminopurine and 0.1 mg/L indolyl acetic acid. Two days after inoculation, explants were transferred to the same medium supplemented with 500 mg/L cefotaxime and 75 mg/L kanamycin. Regeneration shoots were cut and grown on MS medium with antibiotics.

Plants were propagated on MS medium supplemented with 30 g/L sucrose, 0.8 wt/vol agar (Panreac) and 0.3 mg/L indole-3-butyric acid. In vitro cultures were incubated at 24±1° C. with a 12-16-d photoperiod, with mixed cool white and red light (Cool White and Gro-Lux fluorescent lamps) at a light intensity of 40 μmol s-1 m-2. After root development, plantlets were transferred to 9 cm pots with sterilized soil (1:3 wt/wt mixture of sand and peat). Potted plants were placed in the greenhouse at 22±2° C. under neutral day conditions (12 h light/12 h dark; 150 μmol s-1 m-2) and 75% relative humidity.

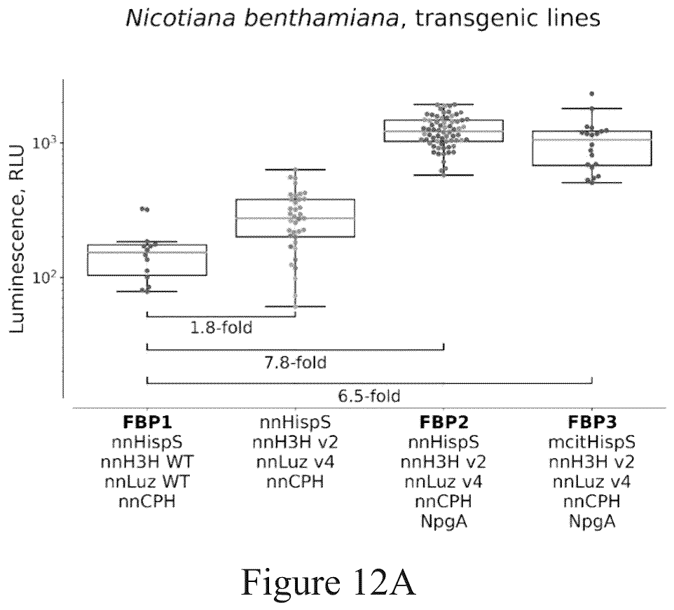

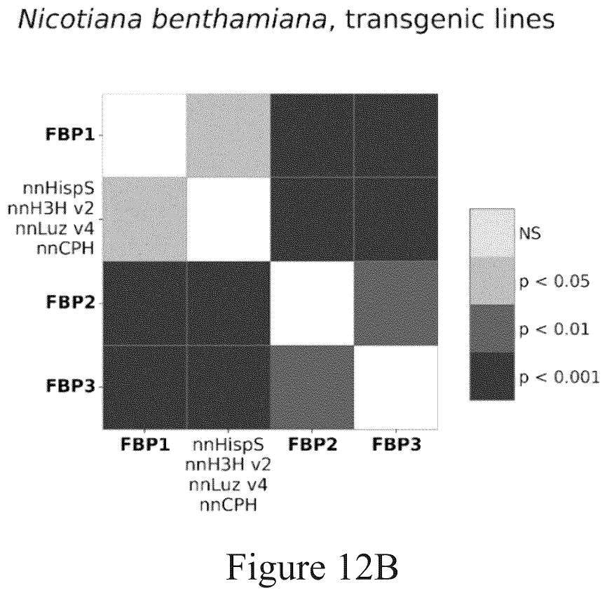

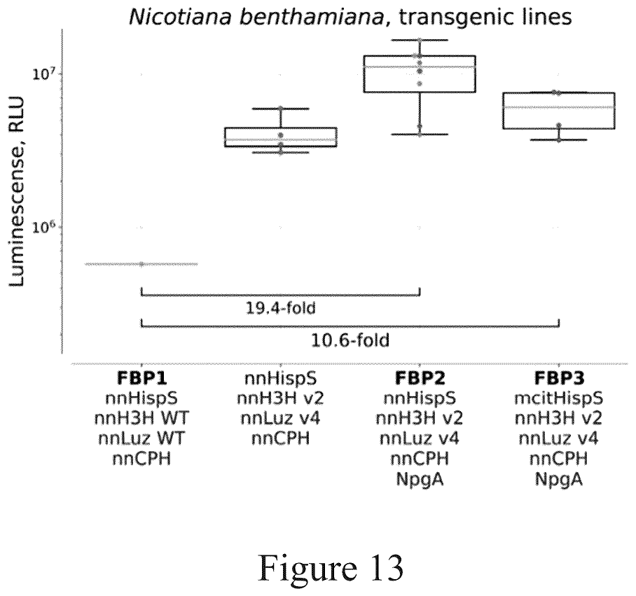

To genotype the transgenic plants, an Eppendorf tube with 100 mg of leaf material was placed in liquid nitrogen and then material was homogenized with pestle. The genome DNA was extracted using the ExtractDNA Blood kit (Evrogen) according to the manufacturer's protocol, and direct PCR was performed on each inserted gene. In transgenic plants produced by this method, greater bioluminescence was produced in leaves by expression of nnH3H_v2/nnLuz_v4 relative to their unmodified counterparts. This was evident in both 3-week-old (FIGS. 12A-12B) and 9-week-old (FIG. 13) transformed plants.

### Example 7. Enhanced Expression of Bioluminescence in Mammalian Cells in the Presence of Caffeic Acid

In different sets of experiments, HEK293NT or HEK293 cell lines were seeded in 96-well plates (ibidi, ibiTreat μ-Plate 96 Well Black). Cells were transfected with a mixture of the five plasmids that encoded variants of nnLuz, and nnH3H, along with nnHispS or mcitHispS, nnCPH, and NpgA by PolyFect Transfection Reagent (QIAGEN) according to the manufacturer's protocol, using 2 μl PolyFect per well and a mixture of plasmids containing 44 ng of plasmid expressing nnLuz, and equivalent amounts of the other four plasmids according to their size. Transfected cells were grown in DMEM (PanEco) supplemented with 10% fetal bovine serum (HyClone), 4 mM L-glutamine, 10 U/ml penicillin and 10 g/ml streptomycin, at 37° C. and 5% CO2. Twenty-four hours after transfection, the medium was changed to 25 μM or 100 μM caffeic acid solution (0.1% DMSO in DPBS) and luminescence was imaged for 23 minutes by IVIS Spectrum CT (PerkinElmer) at 37° C. every 9 seconds with open filter, binning of 16 and exposure of 5 seconds. The number of independent replicates is included in the figure's legends.

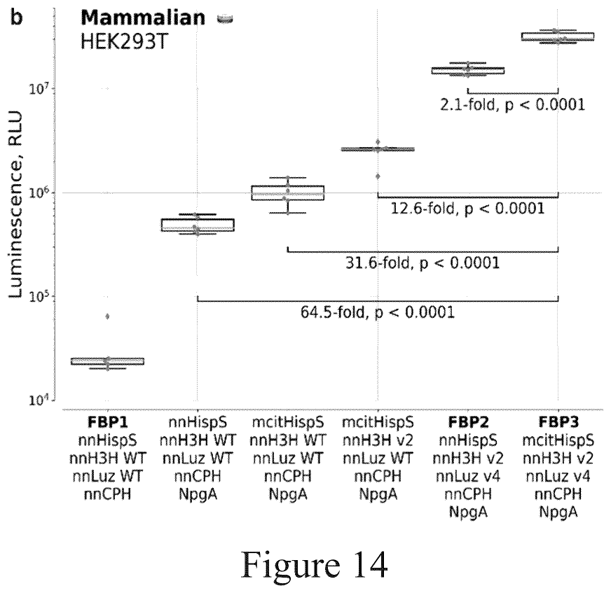

In the gene set expressing nnHispS, nnCPH, and NpgA, the combination of nnH3H_v2 and nnLuz_v4 produced 30.7-fold greater luminescence compared with nnH3H_WT and nnLuz_WT. In similar gene sets expressing mcitHispS instead of nnHispS, nnH3H_v2 and nnLuz_v4 produced 31.6-fold greater luminescence. nnH3H_v2 produced 2.5-fold greater luminescence compared with nnH3H_WT in a gene set expressing nnLuz_WT, mcitHispS, nnCPH, and NpgA (FIG. 14).

### Example 8. Modified Luciferase Shows Greater Enzymatic Activity

To quantify the activity of nnLuz_v4 relative to the parental nnLuz_WT, genetic fusions were made with the HiBiT tag. The HiBiT tag is a small bioluminescent marker that may be appended onto other proteins, thereby providing a mechanism for measuring relative protein concentration. By fusing the tag onto the C-terminus of luciferase, variability in protein concentration can be normalized across independent samples.

Yeast strains expressing fusions of nnLuz_WT and nnLuz_v4 with HiBiT were inoculated into YPD medium and cultured at 30° C. with 220 rpm for 18-20 hours. Then 4 ml of each yeast suspension were centrifuged at 5000 g (4° C.) and resuspended in 400 μL of 100 mM MOPS (pH=7.5), 4 mM EDTA, 2 mM TCEP, 1 mM PMSF. Afterwards, 50-100 pg of 200 μm zirconium beads (Ops Diagnostics) and one glass bead (d=5 mm) were added to each sample. The samples were treated by bead mill homogenizer (TissueLyser LT, Qiagen) at 13,000 rpm at 4° C. for 30 min with pauses. Samples were then centrifuged for 15 min at 4° C. at 10,000 rpm. Lysates were filtered through Spin-X centrifuge tube filter 0.22 μm (Costar) by centrifugation at 13,000 rpm at 4° C. for 5 min.

Samples were diluted 25 fold in the same buffer as used for lysis and concentrated to a volume of 200 μL using Amicon Ultra-15 Centrifugal Filter Unit 10 kDa (Millipore). Then 3.5 μL of lysates were added to 72.5 μL of 0.2 M sodium phosphate buffer with 0.5 M Na2SO4, pH=8, and transferred to black 96-well plate. 25 μL of fungal luciferin (4% DMSO, 0.4% DDM, 40 mM TGA) were added to samples to final concentration 0.0244-50 PM. Luminescence was measured in Tecan Spark plate-reader with exposure of 500 ms for 15 min. Activity of each variant was normalized to the expression level of HiBiT, as quantified with HiBiT lytic reagent (Promega N3030).

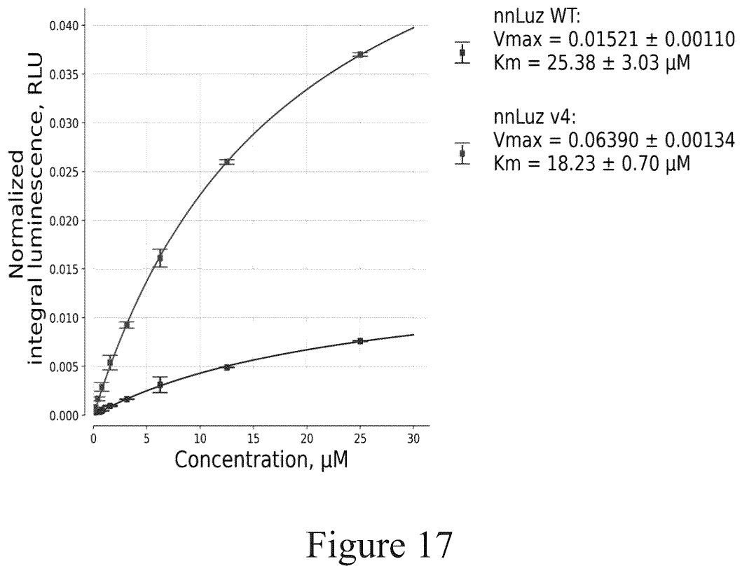

To quantify luminescence, intensity values of the wells were normalized by the integral values for HiBiT luminescence kinetics. Then, the integral signal for each sample was calculated. Fitting of Michaelis-Menten model was performed with scipy package for Python (version 1.10.1), using Michaelis-Menten equation: v=Vmax*[S]/KM+[S]. The resulting fitted model showed that nnLuc_v4 has a 4.2-fold higher Vmax with similar Km (FIG. 17).

The illustrations and examples provided herein are for explanatory purposes and are not intended to limit the scope of the appended claims.

### REFERENCES

1. Mitiouchkina, T. et al. Plants with genetically encoded autoluminescence. Nat. Biotechnol. (2020) doi:10.1038/s41587-020-0500-9.
2. Shakhova, E. et al. An improved pathway for autonomous bioluminescence imaging in eukaryotes. (2024) doi:10.1038/s41592-023-02152-y.
3. Kotlobay, A. et al. Genetically encodable bioluminescent system from fungi. (2018) doi: 10.1073.pnas.18036 15115.

We claim:

1. A method for producing bioluminescence comprising,
providing an isolated nucleic acid encoding a luciferase defining an amino acid sequence at least 90% identical to positions 40-267 of an amino acid sequence identified at SEQ ID NO: 1, the amino acid sequence comprising at least one amino acid substitution selected from T99P, T192S, A199P; and
incorporating the isolated nucleic acid into a transgenic organism capable of biosynthesizing 3-hydroxyhispidin.

2. The method of claim 1, wherein the isolated luciferase defines an amino acid sequence comprising amino acid substitutions T99P, T192S, and A199P.

3. An isolated hispidin hydroxylase defining an amino acid sequence at least 90% identical to the amino acid sequence identified at SEQ ID NO: 5, the amino acid sequence comprising at least one amino acid substitution selected from D37E, V181I, A183P, S323M, M385K.

4. The isolated hispidin hydroxylase of claim 3, wherein the amino acid sequence comprises amino acid substitutions D37E, V181I, S323M, and M385K.

5. A nucleic acid encoding the isolated hispidin hydroxylase of claim 3 or 4.

6. A cell or organism comprising the isolated hispidin hydroxylase of claim 3 or 4.

7. The cell or organism of claim 6, wherein the organism is defined as a plant.

8. An isolated hispidin hydroxylase defining an amino acid sequence identified at SEQ ID NO: 6.

9. A method of producing a hispidin hydroxylase comprising,
providing an isolated nucleic acid encoding a hispidin hydroxylase defining an amino acid sequence at least 90% identical to an amino acid sequence identified at SEQ ID NO: 5, the amino acid sequence comprising at least one amino acid substitution selected from D37E, V181I, A183P, S323M, M385K, and
integrating the isolated nucleic acid into a genome or an extrachromosomal element of a cell.

10. The method of claim 9 where the isolated nucleic acid encodes the hispidin hydroxylase comprising amino acid substitutions D37E, V181I, S323M, and M385K.

11. An isolated luciferase defining an amino acid sequence at least 90% identical to an amino acid sequence identified at SEQ ID NO: 1, the amino acid sequence comprising at least one amino acid substitution selected from T99P, T192S, A199P.

12. The isolated luciferase of claim 11, wherein the amino acid sequence comprises amino acid substitutions T99P, T192S, and A199P.

13. The isolated luciferase of claim 11, wherein the amino acid sequence comprises at least one amino acid substitution selected from R2S, I3N, I3S, N4T, F11L, E12D, I63T.

14. The isolated luciferase of claim 11, wherein the amino acid sequence comprises amino acid substitutions I3S, N4T, F11L, I63T, T99P, T192S, and A199P.

15. An isolated luciferase defining an amino acid sequence as identified at SEQ ID NO: 3.

16. A nucleic acid encoding the isolated luciferase of claim 11 or 12 or 13 or 14 or 15.

17. A cell or organism comprising the isolated luciferase of claim 11 or 12 or 13 or 14 or 15.

18. The cell or organism of claim 17, wherein the organism is defined as a plant.

19. An isolated luciferase defining an amino acid sequence at least 90% identical to positions 40-267 of an amino acid sequence identified at SEQ ID NO: 1, the amino acid sequence comprising at proline at position 199.

20. A cell or organism comprising the isolated luciferase of claim 19.
